(19) United States
(12) Patent Application Publication
(10) Pub. No.: US 2021/0220412 A1
(43) Pub. Date: Jul. 22, 2021

(54) METHODS OF DIAGNOSING AND TREATING CANCER BY DETECTING AND MANIPULATING MICROBES IN TUMORS

(71) Applicant: LOS ANGELES BIOMEDICAL RESEARCH INSTITUTE AT HARBOR-UCLA MEDICAL CENTER, Torrance, CA (US)

(72) Inventors: Delphine J. LEE, Santa Monica, CA (US); Caiyun XUAN, Santa Monica, CA (US)

(21) Appl. No.: 17/217,198

(22) Filed: Mar. 30, 2021

Related U.S. Application Data
(63) Continuation of application No. 16/189,259, filed on Nov. 13, 2018, now abandoned.
Application No. 16/189,259 is a continuation of application No. 14/980,882, filed on Dec. 28, 2015, now abandoned.
Application No. 14/980,882 is a continuation of application No. 14/145,853, filed on Dec. 31, 2013, now abandoned.
(60) Provisional application No. 61/766,501, filed on Feb. 19, 2013.

(51) Int. Cl. A61K 35/741; C12Q 1/6886; C12Q 1/689

(52) U.S. Cl. CPC A61K 35/741; C12Q 1/6886; A61K 2035/115; C12Q 2600/158; C12Q 1/689

(57) ABSTRACT

In some embodiments, methods of determining that a subject is likely to have cancer are provided. Such methods may include amplifying a microbial DNA sample in a test sample obtained from the subject to determine an amount of microbial DNA in the test sample, wherein the amount of microbial DNA is determined by an amplification or sequencing technique; and determining that the subject is likely to have breast cancer when there is a significantly decreased level of microbial DNA in the test sample when compared to a level of microbial DNA in a control sample. In other embodiments, methods of treating cancer (e.g., breast cancer) are provided. In one aspect, such methods include administering a therapeutically effective dose of a probiotic organism via ductal lavage to a subject suffering from the breast cancer.

### CROSS REFERENCE TO RELATED APPLICATIONS

This application is a continuation of U.S. patent application Ser. No. 16/189,259, filed Nov. 12, 2018, which is a continuation of U.S. patent Ser. No. 14/980,882, filed Dec. 28, 2015, which is a continuation of U.S. patent application Ser. No. 14/145,853, filed Dec. 31, 2013, which claims the benefit of U.S. Provisional Patent Application No. 61/766,501, filed Feb. 19, 2013. The entire contents of the foregoing applications are incorporated herein by reference, including all text, tables, sequence listings and drawings.

### BACKGROUND

One in eight women will be diagnosed with breast cancer in their lifetime. It is the second leading cause of death in women, with >40,000 deaths annually (Jemal, 2010). Over the past twenty years over 5.5 billion dollars have been spent on breast cancer research. While progress has been made in treatment and screening there are still 40,000 deaths from breast cancer a year in the United States. While genes and radiation are among known breast cancer causes, the origins of a majority of breast cancer cases remain unknown (Madigan, 1995). It is important to understand how these sporadic breast cancers arise in order to develop preventative strategies against this devastating disease. The recent appreciation of the influence of microbiota on human health and disease begs the question of whether microbes play a role in sporadic breast cancers of unknown etiology

Infections and chronic inflammation have been linked to some cancers but studies of infectious causes of breast cancer have been limited to looking for specific viral signatures in invasive cancers. The breast ducts are intimately associated with cutaneous and oral microorganisms during lactation and sexual activity, and could well harbor infectious agents that contribute to carcinogenesis. It would therefore be beneficial to determine whether bacteria play a role in the development of breast cancer.

### SUMMARY

In some embodiments, methods of determining that a subject has cancer or is at higher risk of developing cancer based on the level of microbes present in tumor and control samples are provided. The microbes may be bacteria, viruses, fungi, or any other microscopic organism or a combination thereof. In certain embodiments, the cancer is a hormonally sensitive cancer. In certain embodiments, the hormonally sensitive cancer is breast cancer. Such methods may include amplifying a microbial DNA sample in a test tissue sample obtained from the subject to determine an amount of microbial DNA in the test tissue sample, wherein the amount of microbial DNA is determined by an amplification or sequencing technique; and determining that the subject is likely to have the cancer when there is a level of microbial DNA in the test sample that is significantly different than a level of microbial DNA in a control sample or standard. In a certain embodiment, the microbial DNA is bacterial DNA. In one embodiment, the bacterial DNA is derived from the species Sphingomonas yanoikuyae or Methylobacterium radiotolerans. In the case where the microbial DNA is derived from the species Sphingomonas yanoikuyae, the subject is likely to have the cancer when there is a level of microbial DNA in the test sample that is significantly lower than a level of microbial DNA in a control sample or standard. In the case where the microbial DNA is derived from the species Methylobacterium radiotolerans, the subject is likely to have the cancer when there is a level of microbial DNA in the test sample that is significantly higher than a level of microbial DNA in a control sample or standard.

In other embodiments, the methods of determining that a subject has cancer or is at higher risk of developing cancer may include determining a microbial fingerprint (also referred to as “microbiome signature”) in a test sample obtained from the subject. In such embodiments, the microbial fingerprint includes one or more test levels of microbial DNA from one or more microbial species or one or more microbial genera. In some aspects, the subject is determined to likely have cancer (e.g., breast cancer) when the one or more test levels of the microbial fingerprint are significantly different from that of a control sample or standard. In some aspects, the one or more microbial species or genera include Sphingomonas and related species (e.g., Sphingomonas yanoikuyae), Methylobacterium and related species (Methylobacterium radiotolerans), or both. In such aspects, the subject is likely to have cancer when (i) a level of Sphingomonas (e.g., Sphingomonas yanoikuyae) microbial DNA is significantly lower than the level in the control sample; (ii) a level of Methylobacterium (Methylobacterium radiotolerans) microbial DNA is significantly higher than the level n the control sample; or (in) both (i) and (n).

In other embodiments, methods of treating a cancer (e.g., breast cancer) are provided. In one embodiment, such methods include administering a therapeutically effective dose of a probiotic organism to a subject suffering from the cancer. In certain embodiments, the cancer may be breast cancer such as a hormone-sensitive cancer. In other embodiments, the probiotic organism is administered via ductal lavage. In another embodiment, methods of treating cancer may include amplifying a microbial DNA sample in a test tissue sample obtained from the subject to determine an amount of microbial DNA in the test tissue sample, wherein the amount of microbial DNA is determined by an amplification or sequencing technique; and administering a probiotic organism to the subject when there is a significantly decreased amount of microbial DNA in the test sample when compared to an amount of microbial DNA in a control sample; wherein the probiotic organism is administered at a therapeutically effective dose. In certain embodiments, the microbial DNA is bacterial DNA. In one embodiment, the bacteria DNA is from a bacterium that is derived from the genus Sphingomonas. In one embodiment, the bacterial DNA is from a bacterium that is derived from the species Sphingomonas yanoikuyae.

In other embodiments, methods of stimulating an increased immune response in a diseased tissue are provided. Such methods may include administering a therapeutically effective dose of a probiotic organism to a subject containing the diseased tissue.

### BRIEF DESCRIPTION OF THE DRAWINGS

FIG. 1 shows that bacterial DNA is present in the vicinity of the breast ductal epithelium. Bacterial 16S ribosomal DNA was detected using fluorescence in-situ hybridization (FISH). Serial sections of FFPE tissues from a breast cancer patient were hybridized with the 16S-specific probe EUB338, or the control probe NONEUB338 as indicated. Images are shown at 40× magnification, with scale bars representing 20 microns.

FIG. 2 illustrates a decrease in bacterial 16S ribosomal DNA in a group of samples that includes both ER+ and ER− breast tumor tissue samples (“Tumor”) versus healthy breast tissue (“Healthy”) and matched normal tissue (“Matched Normal”). Total genomic DNA (gDNA) was extracted from formalin-fixed paraffin-embedded (FFPE) tissues. Copy numbers of the 16S gene were determined using quantitative PCR (qPCR) and normalized by the total gDNA yield. Significance was determined when p<0.05 using Kruskal-Wallis ANOVA followed by Dunn's Multiple Comparison post-test.

FIGS. 3A and 38 illustrate that the decrease in bacterial 16S ribosomal DNA in a group of samples that includes both ER+ and ER− breast tumor tissue samples (“Tumor”) correlates with advanced staging in patients with breast cancer as compared to matched normal samples (“Matched normal”). The amounts of bacterial DNA in breast cancer tissues with the indicated staging were quantified using qPCR. Significance was determined when p<0.05 using Cuzick's trend test.

FIGS. 4A and 43 show the composition of the microbiota at the phylum level in A) matched normal and B) tumor tissues from 20 breast cancer patients. Proteobacteria, Firmicutes, Actinobacteria, Bacteroidetes and Verrucomicrobia were the richest phyla across all samples. Each bar represents 100% of the bacteria detected in a given sample.

FIG. 5 illustrates the abundance of the organism Sphingomonas yanoikuyae in matched normal and breast cancer tissue. A significant reduction in abundance of S. yanoikuyae was found in tumor tissue compared with matched normal adjacent tissue (p<0.01),

FIG. 6 illustrates the abundance of the organism Methylobacterium radiotolerans in matched normal and breast cancer tissue. A significant increase in abundance of M. radiotolerans was found in tumor tissue compared with matched normal adjacent tissue (p=0.01).

FIG. 7 illustrates that antibacterial response genes are down-regulated in breast cancer tissues. Expression levels of antibacterial response genes were analyzed from seven breast cancer patients using total RNA and a PCR array specific for the genes. Expression levels were normalized to a normal adjacent breast tissue sample from a breast cancer patient.

FIG. 8 illustrates a computerized model of the human breast duct as described in Going et al (Going, 2004).

FIGS. 9A and 9B illustrate the process for obtaining a ductogram.

FIG. 9A shows the instillation of fluid into a duct during ductal lavage. FIG. 9B shows a ductogram without extravasation in a woman who has undergone a previous core biopsy for microcalcifications.

FIGS. 10A and 10B illustrate a histological analysis of a breast tissue with ductal carcinoma in situ (MIS). FIG. 10A illustrates DCIS marked by dye from neoadjuvant DCIS study administered by ductal lavage. FIG. 10B is an enlargement of FIG. 10A showing how liquid dye is able to pass through and around DCIS.

FIG. 11 illustrates the identification of bacterial genera present in breast ductal fluid of two normal subjects (Donor 1 and Donor 2). Bacterial diversity in samples from two donors was characterized. Briefly, genomic DNA (gDNA) was isolated from the indicated samples. Purified gDNA was used as a template for PCR detection of the 16S bacterial rDNA gene. PCR products were visualized on an agarose gel, excised and cloned into TOPO cloning vectors. Resulting colonies were sequenced using primers specific for the 16S rDNA gene. Sequences were assigned to bacterial genera based on the Ribosomal Database Project (RDP).

FIG. 12 is a gel illustrating that microbial DNA may be extracted from saline diluted bacteria that are obtained by swabbing the forearm and mouth and are stored at either 4° C. or −80° C.

FIG. 13 shows that Natural Killer T cells (NKT cells) are present in breast tissue from a healthy donor. T cells were isolated from breast tissue using cell foam matrices in media supplemented with IL-2 and IL-15 over the course of three weeks. Harvested T cells were double-labeled for flow cytometry with antibodies recognizing CD3 (anti-CD3-FITC) and invariant TCR (anti-Vα24Jα18-PE). The gated values represent the percentage of double-positive (NKT) cells in each sample.

FIG. 14 is a table providing a summary of clinical data for breast cancer patients used in microbial dysbiosis studies according to the examples below.

FIGS. 15A and 15B illustrate a survey of microbial communities residing in breast tissue from breast cancer patients. FIG. 15A is a pie chart showing the combined distribution at the phylum level in paired normal and breast tumor tissue (n=20). FIG. 15B is a bar graph illustrating the number of operational taxonomic units (OTUs) found in each community (n=20). OTUs found in paired normal adjacent tissue are represented by the solid black bar and OTUs found in tumor tissue are represented by the dark grey bar (p=0.2027).

FIGS. 16A and 16B illustrate principle coordinates analysis (PCoA) plots of paired normal and breast tumor samples. FIG. 16A shows PCoA plots of samples categorized based on histopathology (n=20 paired normal samples). FIG. 16B shows PCoA plots of samples categorized based on tumor stage (n=20 tumor only). No clustering among samples was found based on the categories in A and B.

FIG. 17 shows results of eleven OTUs enriched in paired normal or tumor tissue. Prevalence refers to the number of samples in which the indicated OTU was detectable. Paired Student's t-tests were used to determine differences in abundances of OTUs (n.d.=not detectable).

FIG. 18 illustrates the number of OTUs found in microbial communities residing in paired normal and tumor tissue from patients with ER-positive breast cancer. The top panels show bar graphs of Sphingomonadaceae family abundance (top left panel; p=0.0079), Sphingomonas genus abundance (top center panel; p=0.0258), and Sphingomonas yanoikuyae species abundance (top right panel; p=0.0097). The bottom panels show bar graphs of Methylobacteriaceae family abundance (bottom left panel; p-0.237), Methylobacterium genus abundance (bottom center panel; p=0.0237), and Methylobacterium radiotolerans species abundance (bottom right panel; p=0.0150). OTUs found in paired normal adjacent tissue are represented by the solid black bars and OTUs found in tumor tissue are represented by the dark grey bars.

FIG. 19 illustrates the relative abundances of commonly found skin bacteria, Staphylococcus (top panels) and Corynebacterium (bottom panels), residing in paired normal and tumor tissue from patients with ER-positive breast cancer (n=20), p-values from Student's paired t-test are shown, with p<0.05 considered significant. Error bars represent mean±standard error of the mean (s.e.m). OTUs found in paired normal adjacent tissue are represented by the solid black bars and OTUs found in tumor tissue are represented by the dark grey bars.

FIG. 20 shows the detection of Sphingomonas specific (p=0.0363) and M. radiotolerans (p=0.2508) specific 16s rDNA in paired normal and breast tumor tissues (n=20). Data represent the average of duplicate values. Data were normalized to expression levels of beta-actin. P-values from Student's paired t-test are shown, with p<0.05 considered significant. Error bars represent mean±s.e.m. OTUs found in paired normal adjacent tissue are represented by the solid black bars and OTUs found in tumor tissue are represented by the dark grey bars.

FIGS. 21A and 21B show the correlation of relative abundances of M. radiotolerans and S. yanoikuyae (n=20). FIG. 21A shows the correlation of relative abundances of M. radiotolerans and S. yanoikuyae found in paired normal adjacent tissue (p=0.0003). FIG. 21B shows the correlation of relative abundances of M. radiotolerans and S. yanoikuyae found in tumor tissue (r=0.8882).

FIGS. 22A-22C show the quantification of bacterial load in tissue from healthy and breast cancer patients. FIG. 22A shows copy numbers of the bacterial 16S gene were compared among healthy (age-matched) (n=23), paired normal (n=39) and tumor tissue (n=39). Healthy specimens were obtained from patients undergoing reduction mammoplasty, with no evidence of breast cancer. Statistical analysis was performed using Kruskal-Wallis nonparametric ANOVA with Dunn's Multiple Comparison post-test. FIG. 22B shows bacterial load in tumor tissue according to clinical staging of the tumor specimen. FIG. 22C shows bacterial load in paired normal tissue from the same patients in FIG. 22B according to clinical staging of the tumor specimen. Statistical analysis was performed using Cuzick's Trend test. AH statistical analyses were considered significant when p<0.05. Data represent the average of duplicate values. Error bars represent mean±s.e.m,

FIG. 23 shows a heat map of gene expression values of antibacterial response genes in healthy and breast cancer tissue. The expression values were generated using non-supervised hierarchical clustering. Healthy specimens were obtained from patients undergoing reduction mammoplasty, with no evidence of breast cancer (n=9).

FIGS. 24A-24C show the expression profiles of antimicrobial response genes in healthy and breast cancer tissue. FIG. 24A shows bar graphs showing the expression levels of microbial sensors including Toll-like receptors 2, 5, 9, 1, 4, and 6 (TLR2, TLR5, TLR9, TLR1, TLR4, and TLR6) and cytoplasmic microbial sensors including NOD receptors 1 and 2 (NOD1 and NOD2). FIG. 24B shows bar graphs showing the expression levels of downstream signaling molecules for innate microbial sensors including CARD6, CARD9, TRAF6, IRAK1, IRAK3, and NFKB1. FIG. 24C shows bar graphs showing the expression levels of antimicrobial response effectors including BPI, IL-12A, MPO, PRTN3. SLPI, and CAMP. Healthy specimens were obtained from three patients undergoing reduction mammoplasty, with no evidence of breast cancer; tumor specimens were obtained from six patients with breast cancer (n=9). Solid white bars represent expression levels in healthy tissue and solid grey bars represent expression levels in tumor tissue, p-values from Student's paired t-test are shown, with p<0.05 considered significant. Error bars represent mean±s.e.m.

### DETAILED DESCRIPTION

The embodiments provided herein relate to methods of diagnosing cancer by quantifying microbes in tumor and control tissues. In some embodiments, the cancer is breast cancer. According to some embodiments, tumor tissue may be compared with the microbiota in paired normal tissue to identify dysbiosis that may be associated with cancer disease state and severity. The microbes may be bacteria, viruses, fungi, and any other microscopic organism or a combination thereof. In one embodiment, the level of microbial DNA such as bacterial DNA is quantified. Certain embodiments relate to methods for treating hormone-sensitive cancers, including estrogen receptor positive breast cancer, by administering a probiotic organism that degrades an organic molecule that includes at least one carbon ring, such as a steroid hormone. Other embodiments relate to methods for decreasing levels of steroid hormones, such as estrogen, in a tissue to prevent or reduce the risk of hormone-related cancers. Additional embodiments relate to methods of stimulating an increased immune response by administering a probiotic organism that contains ligands which are recognized by, and which activate, natural killer T (NKT) cells.

The majority of breast tumors arise from epithelial cells lining the breast ducts. Unlike other epithelial surfaces such as the gut, where the microbiota has been extensively studied, the microbial diversity within the breast duct has not yet been described. The ability to easily sample ductal fluid in vivo coupled with next generation sequencing technology allows for the investigation of the entire microbiome and provides an excellent opportunity to investigate the microbial diversity in the breast of normal subjects as compared with those with ductal carcinoma in situ (DCIS). As described in the Examples below, a comprehensive characterization of the breast duct microbiota may be performed in an effort to investigate the relationship between the human breast duct microbiome in vivo and breast cancer development. An examination of ductal fluid showed that microbes reside in the ducts and the ductal fluid in normal healthy women is different between individuals and between breasts in a given person. A mapping of the microbiome of normal and early cancerous breast ducts may identify microbes including bacteria, viruses, and/or fungi that may contribute to carcinogenesis. This information may be used to predict whether an individual has a risk for cancer or is likely to suffer from cancer, and may also be used to provide preventative therapy for those at risk for developing cancer.

### The Role of Bacteria in Cancer

Microbial influence on human health and disease is a new and rapidly expanding area of research. The role of bacteria and their products (e.g., bacterially secreted proteins or factors) in the tumorigenesis of breast cancer has not been well established. In contrast to most of the studies described herein, many studies suggest that the presence of bacteria increases the risk of developing cancer. Microbes have been linked to diseases as varied as obesity (Turnbaugh, 2006; Turnbaugh 2009A), colon cancer (Kostic, 2011; Castellarin, 2012), and colitis (Mazmanian, 2008A). In obese individuals, the ratio of Firmicutes to Bacteroidetes in the colon is significantly higher than in lean individuals (Turnbaugh, 2006; Ley, 2006). Placing obese individuals on low-fat diets resulted in a decrease in this ratio, though not to the levels seen in lean individuals (Ley, 2006). In colon cancer, the overabundance of a single bacterial species Fusobacterium nucleatum correlates with disease and increased likelihood of lymph node metastasis (Castellarian, 2012). In contrast to the pathogenic nature of Fusobacterium in colon cancer, the bacterium Bacteroidetes fragilis exerts a protective effect against colitis by modulating inflammatory immune responses in the gut (Mazmanian, 2008B).

Additionally, Heliobacter pylori infection is associated with increased risk of gastric adenocarcinoma and mucosa-associated lymphoid tissue (MALT) lymphoma. Several epidemiological studies have confirmed the strong association between H. pylori infection and incidences of both intestinal and diffuse-type gastric adenocarcinoma (Siman, 1997; Uemura, 2001). In fact, broad-spectrum antibiotics that eliminate H. pylori infection are a cure for early stage MALT lymphoma (Isaacson, 2004), suggesting that H. pylori is the primary driver of carcinogenesis. It has been reported that H. pylori infection promotes carcinogenesis via induction of chronic tissue inflammation (Naito, 2002). As an example, cyclooxygenase-2 (COX-2), a molecule found in inflammatory tissues with elevated expression levels in breast, colorectal and other cancers, is upregulated in the host response to H. pylori infection (Juttner, 2003). Further, in studies of lymphocyte-deficient mice, infection with the enteric bacteria Helicobacter hepaticus is sufficient to induce intestinal and breast tumorigenesis (Rao, 2006).

In addition to H. pylori, other bacteria have been associated with various forms of cancer. The bacterium Citrobacter rodentium causes colonic disease in mice by promoting inflammation and mucosal hyperplasia (Luperchio, 2001). Infection with C. rodentium causes adenoma formation in a mouse model of colorectal cancer (Newman, 2001). In humans, there is evidence that carriers of the pathogen Salmonella typhi, which causes typhoid fever, are at a 200-fold increased risk of developing hepatobiliary carcinoma (Caygill, 1995). Similarly, Chlamydia psittaci infection is associated with ocular lymphoma in humans, with C. psittaci-eradicating antibiotic therapy having significant clinical efficacy as a drug (Ferreri, 2004). From these and other recent studies, it is becoming increasingly apparent that both community composition and discrete bacterial species can exert pathogenic effects that encourage disease development.

### The Role of Bacteria in Breast Cancer

Bacteria have also been shown to contribute to breast cancer by production of estrogen-like compounds (Clavel, 2005). Given that high estrogen levels are strongly associated with increased risk of breast cancer (Colditz, 1995), these findings suggest that intestinal bacteria can influence breast tumorigenesis. It has also been suggested that bacteria may contribute to breast cancer by inducing chronic inflammation in the host. Pathogenic H. hepaticus infection can lead to increased expression of the pro-inflammatory cytokine TNF-α (Rao, 2006). In the clinic, elevated levels of TNF-α are associated with poor outcome in breast cancer patients (Bebok, 1994).

Studies of breast tissue during plastic surgical procedures have demonstrated the presence of bacteria, mostly Staphylococcus epidermidis and Propionibacterium acnes, consistent with transmission or migration from the skin (Bartisch, 2011; Thornton, 1988; Ransjo, 1985). Furthermore, both culture-dependent methods as well as a recent study based on pyrosequencing of the 16S ribosomal DNA gene of bacteria indicates complex milk bacterial communities, suggesting the human breast duct is not always sterile (Hunt, 2011). However, despite correlative data suggesting that bacterial infection can influence breast tumorigenesis, no clear causal or protective relationships between bacterial infections and breast cancer have been identified. Additionally, in both animal models and clinical trials, treatment with nonsteroidal anti-inflammatory drugs (NSAIDS) reduces breast cancer incidence and limits invasive pathology of breast tumors, suggesting that chronic inflammation may be a risk factor in breast cancer (Holmes, 2010; Steele, 1994).

Although these studies have shown that increased levels of bacteria may contribute to cancer and inflammation, many of the studies described in the Examples below suggest that presence (or enhanced presence) of certain strains of bacteria may decrease the risk of developing cancer and may play a beneficial role in diagnosing, preventing, and treating cancer and inflammation. Recent advances in next-generation sequencing technologies have led to investigations into the role of microbial communities and their interaction with humans in disease pathogenesis. In the studies described in the Examples below, next-generation sequencing was used to define the bacterial communities present in matched normal and breast cancer tissue. These studies showed that the amount of bacteria in both healthy tissues obtained from disease-free reduction mammoplasty patients (“healthy tissue”) and matched normal tissues from breast cancer patients (“matched normal tissue”) were significantly higher compared with that found in tumor tissues. In addition, the abundance of the organism Sphingomonas yanoikuyae was significantly enriched in matched normal tissues, while the abundance of the organism Methylobacterium radiotolerans was significantly enriched in tumor tissues.

The variability of the studies above, combined with the results described in the Examples below, suggests that the role of bacteria in cancer tumorigenesis does not have a “one-size-fits-all” answer. Rather, its role is specific to many variables including, but not limited to, the type of cancer, the tissue involved and the specific strain or strains of bacteria that are present.

### The Role of Viruses in Cancer

Viral causes of cancer such as Human papilloma virus (HPV) in cervical cancer (Durst, 1983; Munoz, 1992; Schwarz, 1985) and Merkel cell polyomavirus in a type of skin cancer (zur Hausen) have been identified. In fact, anti-viral vaccines to prevent cancer have come into clinical practice (Kautsky, 2002; Suzich, 1995). The role of viruses and cancer may be further complicated by the host. For example, the new human virus xenotropic murine leukemia virus-related virus (XMRV) has been detected in prostate cancer tissues (Dong, 2007; Urisman, 2006), though it is not present in all prostate cancer patients. It is possible that XMRV causes prostate cancer in individuals with a specific immunologic abnormality. Chronic XMRV infection is strongly associated with homozygous mutations in the interferon-regulated antiviral molecule RNaseL, and RNaseL mutations predispose the host to prostate cancer (Dong, 2007; Urisman, 2006). Thus, a patient's genetic predisposition paired with their immune function abnormalities may dictate their susceptibility to a cancer-causing virus.

Moreover, although DNA from human papillomavirus (HPV), most commonly associated with cervical cancer, has been detected by some groups in cancerous breast tissues (Akil, 2008; Heng, 2009; Kroupis, 2006), others have failed to find a link between HPV infection and breast cancer (Gopalkrishna, 1996: Lindel, 2007). The ubiquitous human herpes virus Epstein-Barr virus (EBV) has varying presence in breast cancer cells. While some groups report identification of tumors with up to 50% EBV-positivity (Bonnet, 1999; Fina, 2001; Luqmani, 1995; McCall, 2001), other groups have failed to detect EBV in breast cancer tissues altogether (Glaser, 1998; Lespagnard, 1995).

In contrast to viruses, bacteria in the breast have been studied to a far lesser extent. Several groups have investigated the bacteria responsible for infections stemming from breast implant procedures using culture-based methods (Pittet, 2005). Further, the breast milk of healthy women has been shown to harbor an abundance of bacterial species including commonly found skin bacteria (Hunt, 2011; Cabrera-Rubio, 2012). Bacteria in the breast have been studied in the context of infections and in healthy individuals, but no comprehensive study of bacteria in breast cancer has been reported.

Further studies of viruses in breast cancer are needed to determine and establish viral origins of breast cancer. As described herein, deep sequencing techniques may be used to query all microbes, including viruses, thereby increasing the possibility of identifying a potential new virus that may contribute to breast cancer. Additionally, identification of specific viruses that may contribute to breast cancer or other cancers will provide a method of diagnosing whether a patient is at higher risk of developing cancer or is likely to suffer from cancer based on the presence of that particular virus.

### The Role of Viruses in Breast Cancer

In 1936, Dr. John Joseph Bittner, a geneticist and cancer biologist working at the Jackson laboratory in Bar Harbor Me., established the theory that a cancerous agent or “milk factor” could be transmitted by cancerous mothers to young mice from a virus in their mother's milk. The majority of mammary tumors in mice are caused by mouse mammary tumor virus (MMTV); nonetheless evidence for viral etiologies of human breast cancer has been controversial. Interestingly, MMTV-like gene sequences have been identified in the human breast tumors, with 38% of breast cancer tissue from American women testing positive for MMTV-like genes (Etkind, 2000; Wang, 1998; Wang, 1995). In studies of Australian breast cancer patients, prevalence of MMV-like genes correlated with severity of cancer, with invasive breast cancer tissues expressing higher levels of MMTV-like genes compared to noninvasive breast cancer tissues. Furthermore, MMTV-like genes were rarely found in normal breast tissue. Taken together, these data show that the presence of MMTV-like genes in breast tumors correlates with an invasive phenotype and provides evidence that a virus may be associated with human breast tumorigenesis (Ford, 2003).

The availability of techniques for analyzing the whole microbiome combined with the potential role of bacteria, viruses and other microbes in carcinogenesis allows for the establishment of the bacterial and viral diversity of the breast and the examination of the infectious etiology of breast cancer.

### Diagnosing Cancer or the Risk of Developing Cancer

The embodiments as described herein relate to methods of diagnosing a subject with cancer or determining the subject is at risk for developing cancer by detecting and quantifying microbes in tumors. As referred to herein, the term “microbes” includes bacteria, viruses, and fungi or any other microscopic organism or a combination thereof. Such methods may be used to diagnose any cancer or tumor cell type including bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, glandular cancers and uterine cancer. In addition, the methods may be used to diagnose tumors that are malignant (e.g., primary or metastatic cancers) or benign (e.g., hyperplasia, cyst, pseudocyst, hematoma, and benign neoplasm).

Certain embodiments as described herein arise from the unexpected finding that the level of bacteria in the tumor tissue of a breast cancer patient is lower than the level of bacteria in matched normal or healthy breast tissue. As such, a tissue's level of bacteria may be used to aid in determining whether a tissue is cancerous or malignant and whether the patient is at risk for developing cancer. In some embodiments, the level of a microbe such as a bacterium, virus, and fungus or any other microscopic organism or a combination thereof may be used to determine whether a tissue may be cancerous or malignant and whether the patient likely suffers from or is at risk for developing cancer.

Some embodiments described herein are directed to a method for determining whether a subject likely suffers from or is at risk for developing breast cancer. In one embodiment, the subject likely suffers from a hormone sensitive cancer. Estrogen receptor positive (ER+) breast cancer is an example of a hormone sensitive cancer. Additionally, in certain embodiments, methods for diagnosing other hormone-sensitive cancers are provided. As used herein, the terms “diagnosing,” “determining,” and “predicting” may be used interchangeably.

In some embodiments, the methods described herein may be used to diagnose or determine that a patient is at risk of developing any type of breast cancer based on levels or amounts of one or more bacterium which is differentially present in tumor tissue as compared to a control (e.g., a normal tissue, a paired normal tissue or a control standard). These methods may be used to diagnose or determine a patient's risk of developing breast cancer types or subtypes including, but not limited to, ductal carcinoma in situ (DCIS, or intraductal carcinoma), lobular carcinoma in situ, invasive or infiltrating ductal carcinoma, invasive or infiltrating lobular carcinoma, inflammatory breast cancer, triple-negative breast cancer, paget disease, phyllodes tumor, angiosarcoma, adenocarcinoma, low-grade adenosquamous carcinoma, medullary carcinoma, papillary carcinoma, tubular carcinoma, metaplastic carcinoma, micropapillary carcinoma, or mixed carcinoma.

In other embodiments, the methods described herein may be used to diagnose or determine that a patient is at risk of developing any type of breast cancer based on levels or amounts of one or more bacterium which degrades an organic molecule that includes at least one carbon ring such as a steroid hormone. In certain embodiments, the breast cancer is hormone receptive positive breast cancer. Hormone receptor positive breast cancers that may be diagnosed using the methods described herein include those determined to be estrogen receptor positive (ER+), progesterone receptor positive (PR+), androgen receptor positive (AR+) breast cancer, or any combination thereof. For example, hormone receptor positive breast cancers include, but are not limited to, those breast cancers that are ER+/PR+/AR+; ER+/PR+/AR−; ER+/PR−/AR−; ER−/PR+/AR+; ER−/PR+/AR−; ER−/PR−/AR+; or ER+/PR−/AR+. In one embodiment, the methods described herein may be used to diagnose or determine that a patient is at risk of developing ER+ breast cancer, as described in the Examples below. In certain embodiments, the methods described herein may also be extrapolated to other cancers that are estrogen-sensitive or hormone-sensitive including, but not limited to, prostate cancer, ovarian cancer, endometrial cancer, testicular cancer, uterine cancer, and cervical cancer.

The methods for diagnosing or determining that a subject likely suffers from or is at risk for developing cancer may include a step of quantifying the amount of a microbial analyte including protein, RNA, DNA, or any metabolite. For example, in certain embodiments, the methods of diagnosing or determining that a subject likely suffers from or is at risk for developing cancer may include a step of amplifying and/or quantifying the amount of DNA in a test sample and/or a control sample from a subject or patient suffering from or suspected of suffering from cancer. In some embodiments, the DNA may be bacterial, viral, fungal, or any other type of microbial DNA or a combination thereof. In one embodiment, as described further in the Examples below, the bacterial DNA is from a bacterium which degrades an organic molecule that includes at least one carbon ring such as a steroid hormone and the cancer is breast cancer.

In some embodiments, the methods described herein may optionally include a step that includes extracting a DNA sample from a test sample and/or control sample obtained from the subject prior to amplifying the DNA. The DNA sample may be extracted from a tissue or fluid sample from the subject using any suitable method known in the art, including but not limited to methods which incorporate one or more of the following: an organic extraction or precipitation step (e.g., using chloroform, phenol, ethanol, isopropanol or other organic solvent), a column- or bead-separation step, an enzymatic lysis step, a fluorescence in situ hybridization (FISH) step, and/or a DNA sequencing step (e.g., next-generation sequencing, massively parallel sequencing). In some embodiments, the extraction method may include one or more steps carried out using a commercial kit, such as a QIAamp DNA Kit (Qiagen), a DNeasy Tissue Kit (Qiagen), a MicroPrep Kit (Qiagen), a Quanti-it PicoGreensDNA Reagent Kit (Invitrogen); a ChargeSwitch Kit (Invitrogen), DNAIQ (Promega), ForensicGem (ZyGem), or any other suitable kit available to those skilled in the art.

According to the embodiments described herein, the amount of DNA in the test sample and/or control sample may be determined by any suitable quantitative amplification or qualitative detection or sequencing technique for determining the amount (or level) of DNA in a sample (or extracted DNA sample) which contains genomic DNA from the subject, or microbial DNA or a combination thereof. As used herein, “microbial DNA” refers to bacterial DNA, viral DNA, fungal DNA, and any other DNA from a microscopic organism or a combination thereof. Examples of amplification and detection techniques that may be used in accordance with the embodiments described herein may include, but are not limited to, a quantitative polymerase chain reaction assay (q-PCR), real time PCR, digital PCR, in-situ hybridization, cDNA microarray, or immunohistochemistry/immunofluorescence using an antibody that targets a cell surface protein of S. yanoikuyae. In one embodiment, the bacterial DNA is amplified using the amplification technique, q-PCR. q-PCR may be performed using universal bacterial rDNA primers such as 63F and 355R to detect the copy numbers of bacterial 16S rDNA.

In some embodiments, the quantification techniques may be used to quantify the amount (or level) of a specific type of microbial DNA (i.e., a particular species or strain). In one embodiment, the quantification technique may be used to quantify bacterial DNA from a bacterial organism that is able to degrade an organic molecule that includes at least one carbon ring. Examples of bacteria that may degrade an organic molecule having at least one carbon ring include, but are not limited to, those bacteria of the genera Sphingomonas, Arthrobacter, Achromobacter, Alcaligenes Acidovorax, Bacillus, Brevibactenum, Burkholderia, Chryseobacterium, Cycloclasticus, Janibacter, Marinobacter, Nocardioides, Pasteurella, Polaromonas, Ralstonia, Rhodanobacter, Staphylococcus, Stenotrophomonas, Terrabacter, Xanthamonas, Mycobacteum, Pseudomonas, and Rhodococcus (Seo, 2009). In some embodiments, the bacteria described herein that degrade an organic molecule having at least one carbon ring is from the genus Sphingomonas. In one aspect, DNA from bacteria from the species Sphingomonas yanoikuyae is amplified in accordance with the methods described herein. As referred to herein, the genus Sphingomonas refers to and includes any and all genera within the Sphingomonas genus (i.e., all “sphingomonads”) including, but not limited to, Sphingomonas, Sphingobium, Novosphingobium, Sphingosinicella, and Sphingopyxis.

The quantification techniques described herein may be used to quantify bacterial DNA from any other suitable and relevant bacterial organism. In one embodiment, the quantification techniques may be used to quantify bacteria of the genera Methylobacterium. In one aspect, DNA from bacteria from the species Methylobacterium radiotolerans is amplified in accordance with the methods described herein.

In some embodiments, the organic molecule that may be degraded by one or more of the bacteria described above and that includes at least one carbon ring includes an aromatic molecule. An example of an aromatic molecule is benzene. In certain embodiments, the organic molecule that may be degraded by one or more of the bacteria described above and that includes at least one carbon ring is a steroid hormone molecule that plays a role in the development of hormone-sensitive cancers. Steroid hormone molecules include three six-membered carbon rings and one five-membered carbon ring. Examples of classes of steroid hormones that play a role in the development of hormone-sensitive cancers include, but are not limited to, estrogens, androgens, and progestins. In one embodiment, the steroid hormone molecule that may be degraded by one or more of the bacteria described above is an estrogen molecule. The estrogen molecule may be an estrone, an estradiol, or an estriol. In one embodiment, the estrogen molecule that may be degraded by one or more of the bacteria described above is estradiol.

Other examples of organic molecules that include at least one carbon ring that may be degraded by one or more of the bacteria described above in accordance with the methods described herein include heterocyclic aromatic amines (HAAs) and polycyclic aromatic hydrocarbons (PAHs). PAHs include at least one fused aromatic ring and are chemical products of combustion from coal burners, fuel, cigarette smoke, and various other sources. PAHs have been shown to be carcinogenic and to increase risk for breast cancer in a variety of ways. The most common PAHs are weakly estrogenic (estrogen mimicking), due to interactions with the cellular estrogen receptor (ER). As such, methods for administering a probiotic that includes a species of bacteria that is able to degrade PAHs may be used as a prophylactic treatment in subjects exposed to environmental sources of PAHs to prevent the development of estrogen-related or estrogen-sensitive cancers including, but not limited to, breast cancer, ovarian cancer, and cervical cancer.

In some embodiments, a variety of quantification techniques may be used to determine the level of microbes, such as microbial DNA, from a particular genus or species that are present in a test and/or control sample. Quantification of a particular microbial DNA may be determined by qualitative or quantitative methods that include, but are not limited to, amplification and detection techniques, sequencing techniques, or hybridization techniques or other techniques including, but not limited to, quantitative PCR, real time PCR, digital PCR, in-situ hybridization, cDNA microarrays, or immunohistochemistry/immunofluorescence. In one embodiment, quantitative PCR may be performed using primers specific to the bacterial genus or species to be detected to determine the copy numbers of specific bacterial DNA. In another embodiment, the amount of microbial DNA of a particular genus or species of microbe may be determined using a variety of massively parallel sequencing techniques that include, but are not limited to, pyrosequencing, single molecule real time sequencing, bridge PCR, ion semiconductor sequencing, sequencing by synthesis, sequencing by ligation, and chain termination sequencing (Sanger sequencing).

As used herein, a “subject” refers to a human or animal, including all mammals such as primates (particularly higher primates), sheep, dog, rodents (e.g., mouse or rat), guinea pig, goat, pig, cat, rabbit, and cow. In some embodiments, the subject is a human.

As described above, the methods used to diagnose cancer may include determining an amount of microbes or microbial DNA in a test tissue sample and/or a control sample. The “test sample,” as referred to herein, may include one or more tissue or fluid samples containing tumor cells that are obtained from a subject that has or is suspected of having cancer. The test sample may be obtained from tissues where the cancer has either originated or metastasized in the subject. In one embodiment, the test sample may include a tumor tissue obtained from a post-menopausal woman with breast cancer. In one embodiment, the test sample contains breast tumor cells (e.g., tumor tissue sample or primary culture of breast cancer cells). In one aspect, the test tissue sample may include a plurality of tissue samples that may be compared to a control sample or reference standard as described below in order to study differences between similarly situated populations or groups.

In another embodiment, the test sample may be ductal fluid obtained from the breast ducts of a subject. Breast ducts are lined with a small amount of fluid, the characterization of which has demonstrated the presence of numerous components, including cellular constituents such as ductal epithelial cells and macrophages; serum proteins such as albumin and immunoglobulins; hormones such as estrogens, androgens, progesterone, dehydroepiandrosterone sulfate (DHEAS), and prolactin; growth factors such as epidermal growth factor and transforming growth factor α and other biomolecules such as lipids, cholesterol and lactose (Petrakis, 1986). In some embodiments, the ductal fluid is nipple aspirate fluid (NAF) or ductal fluid obtained by ductal lavage. For example, the test sample may be ductal fluid from an individual with DCIS. In one embodiment, the individual duct contains DCIS. In another embodiment, the individual duct does not contain DCIS, but is from a breast containing other ducts with DCIS. In another embodiment, the test sample may be ductal fluid from a woman that is premenopausal with DCIS. In one embodiment, the test sample may be ductal fluid from a woman considered to be at high-risk for developing breast cancer.

According to some embodiments, the “control sample,” as referred to herein, may include one or more healthy tissue or fluid samples from one or more healthy subjects that do not have cancer. In certain embodiments, the control sample is obtained from the same subject from whom the test sample was obtained. In such embodiments, the control sample may be obtained from an area adjacent to the site from where the test sample was obtained, which may be referred to herein as “matched normal tissue,” “matched adjacent tissue,” “matched healthy tissue,” “paired normal adjacent tissue,” or “paired normal.” In other embodiments, the control sample is obtained from a different subject than from whom the test sample was obtained. In certain embodiments, the control sample may be obtained from the subject from whom the test sample was obtained, from a different subject from whom the test sample was obtained, or a combination thereof. In still other embodiments, the control sample may include samples obtained from a population of different subjects, which may or may not include the subject from whom the test sample was obtained. In some embodiments, the subject from whom the control sample is obtained may or may not have cancer. In still other embodiments, the control sample may include healthy tissue or fluid samples obtained from a population of subjects that have cancer and do not have cancer. In some embodiments, the amount of microbial DNA (e.g., the amount of total microbial DNA or the amount of a particular microbial genus or species DNA) that is measured or quantified in a population or plurality of subjects may be used to establish a reference standard or control standard to which a test sample may be compared. In one embodiment, the control samples are from fluid samples obtained from the breast ducts of normal healthy women.

A test sample and/or control sample may be obtained from any tissue or fluid which contains genomic DNA, microbial DNA or DNA from any other microorganism. As described above, the sample may be obtained from a tumor tissue, an adjacent normal tissue, or healthy tissue; and may be a fresh frozen sample, formalin-fixed paraffin-embedded (FFPE) sample, a primary cell culture, or any other suitable tissue. In certain embodiments, the test and control samples are FFPE tissue samples or fresh frozen samples.

Additionally, the sample may be obtained from a fluid sample including nipple aspirate fluid (NAF) or ductal fluid obtained by ductal lavage. Non operative techniques such as NAF and ductal lavage have been developed to sample the breast fluid. NAF can be obtained from approximately 60% of women, and is the easiest to obtain. However, it is not usually expressed from all of the ducts and its physiology is not understood. It may be representative of the small amount of fluid found in all of the ducts, or it could represent a pathologic process, such as a low grade inflammation present only in some ducts. Previously, the patterns of cytokines in NAF have been compared to that in lavage fluid and they appear to be distinct (Love, 2011). Furthermore, ducts that do not produce NAF are as likely to have atypical cells as ducts that do (Twelves, 2011; Chatterton, 2004; Bhandare, 2005; Chatterton, 2010). The ductal fluid may also be obtained by lavage. Ductal lavage enables sampling of ductal fluid from all women, thus increasing the availability of subjects, avoiding any bias, and ensuring that the normal ductal microbiome is what is reflected. The technique involves local anesthetization of the nipple followed by duct dilation and cannulation. Saline (or another biocompatible fluid) is instilled into the ductal system through the nipple and subsequently recovered, bringing with it epithelial cells and other components of the ductal fluid. Ductal lavage allows minimally invasive sampling of the ductal fluid of individual ducts. In some embodiments, the fluid sample may be a flash frozen sample.

Once the levels of microbial DNA have been determined for the test and/or control samples, the levels of microbial DNA may then be compared between samples or between the test sample and a reference standard or control standard to determine whether the subject has cancer. When a level of microbial DNA is significantly different than a level of microbial DNA in the control (e.g., control sample, reference standard, or control standard), the subject may be determined to be likely suffering from cancer or may be at increased risk of developing cancer (e.g., breast cancer). In certain embodiments, when the level of microbial DNA in the test sample is significantly lower or decreased compared with a control sample or a reference standard, the subject may be determined to have cancer or be at increased risk of developing cancer. In still other embodiments, when the level of microbial DNA in the control sample or the reference standard is significantly higher or increased compared to the level of microbial DNA in a test sample, the subject may be determined to have cancer or be at increased risk of developing cancer.

Alternatively, in other embodiments, when the level of microbial DNA in the test sample is not significantly lower or is comparable to that in the control sample, the subject is not likely to be suffering from cancer. In one embodiment, the microbial DNA is bacterial DNA. In another embodiment, the subject is likely to have breast cancer. In some embodiments, the methods may include a step of determining that the subject has breast cancer when there is a significantly decreased level of bacterial DNA in the test sample when compared to a level of bacterial DNA in a control sample. In some embodiments, the bacterial DNA is from the species Sphingomonas yanoikuyae.

According to certain embodiments as described herein, the level of microbial DNA may be used to determine whether a subject is likely to be suffering from cancer. In some embodiments, when the level of microbial DNA in the test sample is higher or significantly increased compared with a control sample or a reference standard, the subject may be determined to have cancer or be at increased risk of developing cancer. In still other embodiments, when the level of microbial DNA in the control sample or the reference standard is decreased or is significantly lower compared to the level of microbial DNA in a test sample, the subject may be determined to have cancer or be at increased risk of developing cancer. In some embodiments, the microbial DNA is viral DNA. In other embodiments, the microbial DNA is bacterial DNA from the species Methylobacterium radiotolerans. In such embodiments, the method of treating a cancer (e.g., breast cancer) may include providing or administering a therapeutically effective amount of a vaccine or an immunotherapy regimen in a patient suffering from or at risk of developing the cancer. In one embodiment, the vaccine or immunotherapy regimen may include an antigenic protein or protein fragment which stimulates an immune response against M. radiotolerans. Such a vaccine would be preventative similar to the FDA-approved HPV vaccine used in used to prevent cervical cancer according to the current standard of care in normal or high-risk subjects. In one embodiment, an immunotherapy regimen may include a probiotic treatment or treatment regimen, such as the treatments described herein.

As used herein, the term “significantly” or “significant” refers to a result that is statistically significant. In certain embodiments, statistical significance may be determined using any known test used to determine statistical significance. For example, a paired Student's t-test may be used to determine statistical significance. As described herein, a calculated p-value with a threshold of p<0.05 is considered statistically significant. In one embodiment, the calculated p-value of p=0.01 is used as a threshold of statistical significance. For example, in one embodiment, the level of bacterial DNA is considered to be significantly lower if the calculated p-value is at least p=0.01 using a paired Student's t-test. In other embodiments, the term “significantly” or “significant” may be used to refer to a relative comparison between two or more experimental groups that are of interest. For example, if the results (i.e., expression level, quantity of bacteria or other measurable result) obtained from two experimental groups are found to be different by a factor of more than one, then this difference may be referred to as significant. In some embodiments, two or more groups may be significantly different if their experimental results are different by a factor of 2, 3, 4, 5, 6, 7, 8, 9, 10, or greater than 10.

According to some embodiments described herein, once the level of bacterial DNA of a particular bacterial genus or species to be detected has been determined for the test and control samples, the levels may then be compared between the test and control samples. In certain embodiments, if the level of bacterial DNA of a particular bacterial genus or species to be detected in the test sample is decreased or significantly lower than that in the control sample, the subject is likely to be suffering from cancer (e.g., breast cancer). In one embodiment, the subject has breast cancer. In certain embodiments, if the level of bacterial DNA from Sphingomonas genera from a test sample is significantly lower as compared to a control sample, then the subject is likely to be suffering from cancer. In one embodiment, a calculated p-value that is equal to or below a p=0.0363 threshold of statistical significance using a paired Student's t-test is considered to be significantly lower. In one embodiment, the level of bacterial DNA from Sphingomonas yanoikuyae from a test sample is considered to be significantly lower as compared to a control sample. In one embodiment, a calculated p-value that is equal to or below the p=0.0097 threshold of statistical significance using a paired Student's t-test is considered to be significantly lower.

According to certain embodiments, a microbial fingerprint and methods for determining a microbial fingerprint of a test sample from a subject are provided, and may be useful in methods for determining whether the subject may or may not be suffering from cancer (e.g., breast cancer). As such, methods for determining whether a subject has cancer (e.g., breast cancer) are provided, and may include steps including, but not limited to, ascertaining or determining a microbial fingerprint of a test sample obtained from a subject suspected of having the cancer, and determining that the subject is likely to be suffering from the cancer or is not likely to be suffering from the cancer based on the microbial fingerprint as compared to a control sample or standard.

As used herein, the term “microbial fingerprint” describes a panel of microbial DNA measured in a sample obtained from a subject, and includes one or more test levels of microbial DNA from one or more microbial species or one or more microbial genera. The one or more test levels may be differentially present in a cancerous or tumorigenic state. For example, the microbial fingerprint of a test sample may indicate a level of microbial DNA of a particular genus or species that is increased or significantly higher compared to the level of microbial DNA from a different genera or species in the test sample. In some embodiments, the microbial fingerprint of a test sample may indicate a level of microbial DNA from a particular genus or species that is decreased or significantly lower compared to the level of microbial DNA from other genera or species in the test sample. In one embodiment, a microbial fingerprint may include a level of Sphingomonas microbial DNA (including any and all Sphingomonas species), a level of Sphingobium microbial DNA (including any and all Sphingobium species), a level of Methylobacterium microbial DNA (including any and all Methylobacterium species), or a combination thereof. In another embodiment, a microbial fingerprint may include a level of Sphingomonas yanoikuyae microbial DNA, a level of Methylobacterium radiotolerans microbial DNA, or both. In another embodiment, a microbial fingerprint may indicate the overall total microbial population.

The different levels of microbial DNA from various genera or species in the test sample that make up the microbial fingerprint of the test sample may be useful in determining whether a subject may or may not be suffering from cancer.

A microbial fingerprint of a test sample may be determined by quantifying the levels of microbial DNA of various types of microbes (e.g., different genera or species) that are present in the test sample. In some embodiments, the levels of microbial DNA of various genera or species of microbes that are present in the test sample may be determined and compared to that of a control sample or standard. In certain embodiments, if the level of microbial DNA of a particular genus or species in the test sample is decreased or significantly lower than a control sample or standard, the subject is likely to be suffering from cancer (e.g., breast cancer). In some embodiments, the subject is likely to be suffering from cancer if the microbial fingerprint shows the following:
(i). the level of microbial DNA of the genus Sphingobium detected in the test sample is decreased or significantly lower than a control;
(ii). the level of microbial DNA of the genus Sphingomonas detected in the test sample is decreased or significantly lower than a control;
(iii). the microbial DNA of the genus Methylobacterium detected in the test sample is increased or significantly higher than a control; or
(iv). A combination of one or more of (i), (ii), and (iii).

In some embodiments, the subject is likely to be suffering from cancer if the microbial fingerprint shows the following:
(i). the level of microbial DNA of the species Sphingomonas yanoikuyae detected in the test sample is decreased or significantly lower than a control;
(ii). the microbial DNA of the genus Methylobacterium radiotolerans detected in the test sample is increased or significantly higher than a control; or
(iii). a combination of one or both of (i) and (ii).

In certain embodiments, the levels of microbial DNA of various genera or species of microbes that are present in the test sample may be determined and compared between the other various genera or species present in the test sample. In certain embodiments, if the level of microbial DNA of a particular genus or species in the test sample is decreased or significantly lower than the microbial DNA of other microbial genera or species detected in the test sample, the subject is likely to be suffering from cancer (e.g., breast cancer). In one embodiment, if the level of microbial DNA of the genus Sphingobium (i.e., all sphingomonads) is decreased or significantly lower than the microbial DNA of the genus Methylobacterium detected in the test sample, the subject is likely to be suffering from cancer. In one embodiment, if the level of microbial DNA of the species Sphingomonas yanoikuyae is decreased or significantly lower than the microbial DNA of the species Methylobacterium radiotolerans detected in the test sample, the subject is likely to be suffering from cancer.

In certain embodiments, if the level of microbial DNA of a particular microbial genus or species in the test sample is not significantly different or is comparable to the level of microbial DNA of a different microbial genera or species detected in the test sample, the subject is not likely to be suffering from cancer (e.g., breast cancer). In some embodiments, if the level of microbial DNA of the species Sphingomonas yanoikuyae is not significantly different or is comparable to the level of microbial DNA of Methylobacterium radiotolerans, the subject is not likely to be suffering from cancer.

According to certain embodiments, if the level of microbial DNA of a particular microbial genus or species in the test sample has a strong inverse correlation between the level of microbial DNA of a different microbial genera or species detected in the test sample, the subject is not likely to be suffering from cancer (e.g., breast cancer). In one embodiment, if there is a strong inverse correlation between the level of microbial DNA from the species Sphingomonas yanoikuyae and Methylobacterium radiotolerans in the test sample, the subject is not likely to be suffering from cancer. In one embodiment, a calculated p-value that is equal to or below the p=0.0003 threshold of statistical significance using a paired Student's t-test is considered to be an indication of a strong inverse correlation. In certain embodiments, if there is not a strong inverse correlation between the level of microbial DNA from the species Sphingomonas yanoikuyae and Methylobacterium radiotolerans in the test sample, the subject is likely to be suffering from cancer.

In certain embodiments, the amount of total microbial DNA in a test sample may be useful in determining whether a subject may or may not be suffering from cancer. In some embodiments, the copy number of 16S ribosomal DNA (rDNA) may be determined to quantify the total microbial DNA in a sample (e.g. total bacterial counts in a sample). In certain embodiments, a qPCR analysis may be performed to enumerate 16S rDNA copy numbers. In some embodiments, if the amount of total microbial DNA in the test sample is decreased or is significantly lower compared to the amount of total microbial DNA in a control sample, the subject is likely to be suffering from cancer (e.g. breast cancer). In certain embodiments, the control sample may be healthy tissue from patients with no evidence of breast cancer and a calculated p-value that is equal to or below the p<0.01 threshold of statistical significance using a paired Student's t-test is considered to be significantly lower. In certain embodiments, the control sample may be paired normal tissue and a calculated p-value that is equal to or below the p<0.001 threshold of statistical significance using a paired Student's t-test is considered to be significantly lower. In certain embodiments, if the amount of total microbial DNA in the test sample is not decreased or is not significantly lower compared to the amount of total microbial DNA in a control sample, the subject is not likely to be suffering from cancer (e.g. breast cancer).

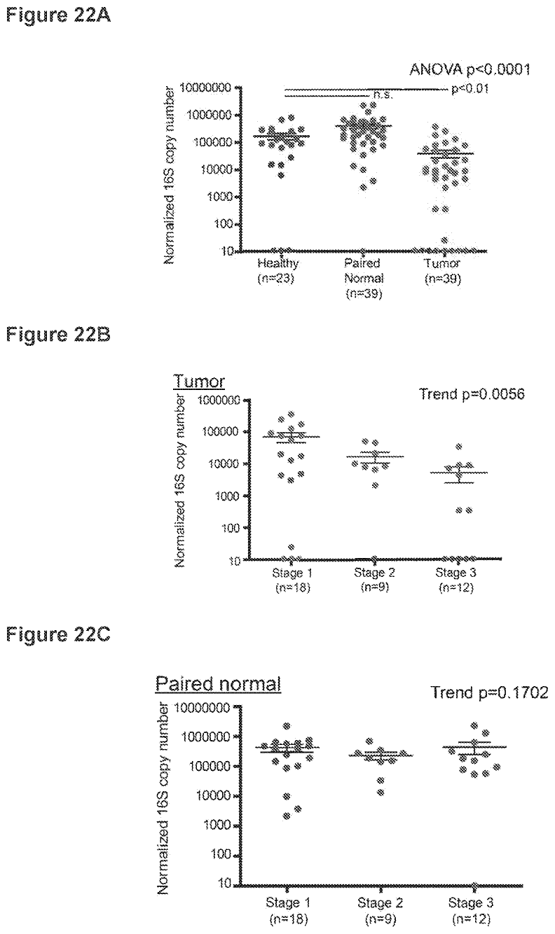

In certain embodiments, the amount of total microbial DNA in a test sample may be useful in determining the severity of cancer of the tumor, such as the particular stage of cancer (e.g. breast cancer stage). In certain aspects, the amount of total microbial DNA is inversely proportional to more advanced stages or cancer (See FIG. 22B)

Identification and quantification of the overall composition of the microbes present and/or the levels of microbial DNA of different types of microbes present in a test sample (e.g., tumor and/or control samples) may, in addition to the amplification techniques described herein, be performed using a suitable sequencing technique, including a variety of high-throughput (next generation) sequencing techniques that include, but are not limited to, pyrosequencing, single molecule real time sequencing, bridge PCR, ion semiconductor sequencing, sequencing by synthesis, sequencing by ligation, and chain termination sequencing (Sanger sequencing). In certain embodiments, the composition of the microbes may be determined using the next generation pyrosequencing sequencing platform the MiSeq System (Illumina, Inc.). Briefly, genomic DNA may be amplified using fusion primers targeting the bacteria 16S V4 rDNA with indexing barcodes. Samples may be amplified with two differently barcoded V4 fusion primers and pooled for sequencing on the Illumina Miseq. Sequences may be quality filtered and demultiplexed using Quantitative Insights Into Microbial Ecology (QIIME) (Caporaso, 2010) and custom scripts with exact matches to the supplied DNA barcodes. Resulting sequences may then be searched against the Greengenes reference database of 16S sequences (DeSantis, 2006) and clustered at by uclust (Edgar, 2010). In one embodiment, this technique may be used to determine the level of Sphingomonas yanoikuyae in breast cancer test tissue compared with normal control tissue.

In other embodiments, the 454/Roche sequencing platform is used to analyze microbial DNA such as bacterial 16S rDNA. Briefly, the samples may be prepared using degenerate PCR primers that have been developed for variable regions within the 16S rDNA gene. For example, regions V1-V3 and V3-V5 may be used according to the protocol adapted by the Human Microbiome Project. PCR may be performed on the samples using 96 versions of a primer pair, the PCR products may be pooled, and a single library may be constructed per variable for 454 sequencing.

In another embodiment, high-throughput sequencing technology may be used to analyze the diversity of the microbial genome of the test and/or control samples. For example, the Solexa/Illumina HiSeq platform may be used. In certain embodiments, this platform may be used to analyze the bacterial, viral, and fungal genera and species present in test and/or control samples. Additionally, in some embodiments, whole genome amplification using the multiple displacement amplification (MDA) approach may also be utilized. MDA uses D29 DNA polymerase to amplify whole genomes (GenomiPhi DNA amplification kit by Amersham Biosciences) (Dean, 2001; Detter, 2002). In certain embodiments, RNA-seq may be performed to identify the microbes, including RNA viruses, present in the test and/or control samples.

In some embodiments, techniques may also be used to determine the histological location of bacteria in tissue. In one embodiment, the histological location of bacteria may be determined in a test sample and control sample. For example, fluorescence in situ hybridization (FISH) using a probe for bacterial ribosomal DNA such as 16S rDNA may be performed on test samples and control samples. A universal bacterial probe such as EUB338 may be used to directly identify and locate the bacterial 16S rDNA. The probes may contain a fluorescence label that can be visualized using a microscope such as the Leica LMD7000 microscope. Other methods known in the art (e.g., immunoassays or other hybridization assays) may also be used to visualize the histological location of bacteria in tissue.

### Treatment of Cancers

In certain embodiments, the methods described herein may be used to treat cancers such as those cancers described in detail above. According to some embodiments, the treatment methods may be methods for treating or optimally treating any type or subtype of breast cancer including, but not limited to, ductal carcinoma in situ (DCIS, or intraductal carcinoma), lobular carcinoma in situ, invasive or infiltrating ductal carcinoma, invasive or infiltrating lobular carcinoma, inflammatory breast cancer, triple-negative breast cancer, paget disease, phyllodes tumor, angiosarcoma, adenocarcinoma, low-grade adenosquamous carcinoma, medullary carcinoma, papillary carcinoma, tubular carcinoma, metaplastic carcinoma, micropapillary carcinoma, or mixed carcinoma. According to other embodiments, the treatment methods may be methods for treating or optimally treating hormone sensitive cancers. For example, the hormone-sensitive cancer that is treated according to the embodiments described herein is an estrogen-receptor positive (ER+) breast cancer.

The method of treating or optimally treating cancers includes a step of administering a therapeutically effective amount or dose of a probiotic organism to a subject suffering from cancer. The probiotic organism as referred to herein may include a bacterium that degrades an organic molecule that has at least one carbon ring as described in detail above. In some embodiments, the probiotic organism includes at least one bacterial species from the genus Sphingomonas. In one aspect, the probiotic includes bacteria from the species Sphingomonas yanoikuyae.

The organic molecule that has at least one carbon ring may be a steroid hormone molecule that plays a role in the development of hormone-sensitive cancer as previously described. In some embodiments, the steroid hormone molecule is an estrogen molecule, such as estrone, estradiol, and/or estriol. In one aspect, the estrogen molecule is estradiol.

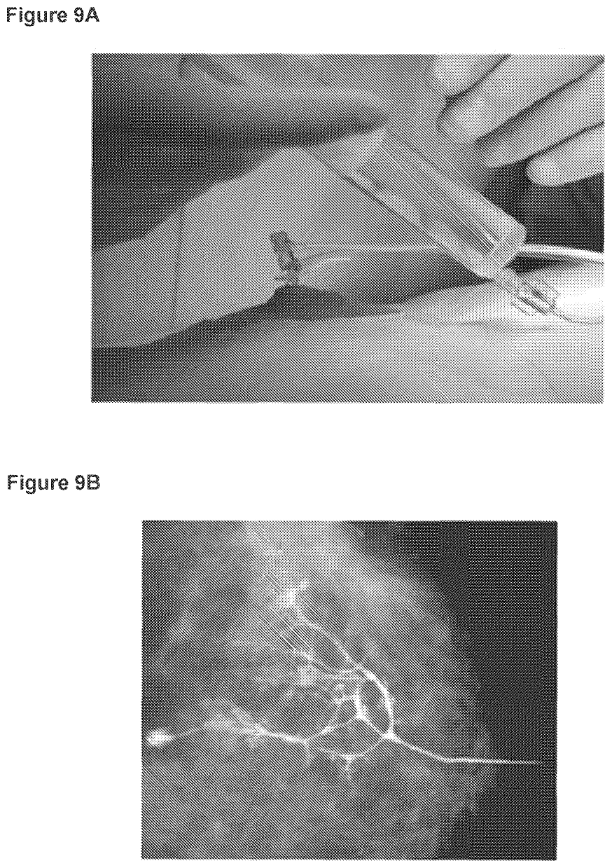

The probiotic organism as described herein may be administered by any suitable route of administration, alone or as part of a pharmaceutical composition. A route of administration may refer to any administration pathway known in the art, including but not limited to aerosol, enteral, nasal, ophthalmic, oral, parenteral, rectal, transdermal (e.g., topical cream or ointment, patch), or vaginal. “Transdermal” administration may be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrastemal, intrathecal, intratumoral, intrauterine, intravenous, subarachnoid, subcapsular, subcutaneous, transmucosal, or transtracheal. In some aspects, an intratumoral administration may be accomplished in concert with a radiologically-assisted technique (e.g., XRay, CT scan, MRI, PET) to visualize the location of the cancer. In one embodiment, the probiotic organism is administered via ductal lavage (see FIG. 9A). Ductal lavage is a minimally invasive technique that may be used to introduce probiotic organisms into the breast.

In some embodiments, the therapeutically effective amount of probiotic organisms is an “effective amount,” “therapeutically effective concentration” or “therapeutically effective dose.” In some embodiments, the therapeutically effective amount is the lowest dose of probiotic organism required to maintain a therapeutic benefit to the subject. In some embodiments, the precise therapeutically effective amount or effective amount is an amount of a probiotic organism that will yield the most effective results in terms of efficacy of treatment in a given subject or population of cells. This amount will vary depending upon a variety of factors, including but not limited to the characteristics of the probiotic organism (including activity, strain, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further, an effective or therapeutically effective amount may vary depending on whether the probiotic organism is administered alone or in combination with another organism, compound, drug, therapy or other therapeutic method or modality. One skilled in the clinical and pharmacological arts will be able to determine an effective amount or therapeutically effective amount through routine experimentation, namely by monitoring a cell's or subject's response to administration of the probiotic organism and adjusting the dosage accordingly. For additional guidance, see Remington: The Science and Practice of Pharmacy, 21^{st} Edition, Univ. of Sciences in Philadelphia (USIP), Lippincott Williams & Wilkins, Philadelphia, Pa., 2005, which is hereby incorporated by reference as if fully set forth herein.

In certain embodiments, the therapeutically effective dose of the probiotic organism is a dose sufficient to maintain a level of bacterial DNA in a test sample at a level that is approximately equal to a level of bacterial DNA in a control sample. In one embodiment, the therapeutically effective dose of the probiotic organism is a dose sufficient to maintain a level of bacterial DNA in a test sample at a level that is greater than a level of bacterial DNA in a control sample.

In other embodiments, the method of optimally treating cancer in a subject as described herein includes a step of amplifying a microbial DNA sample in a test sample from the subject to determine an amount of microbial DNA. In certain embodiments, the microbial DNA is bacterial DNA and the cancer is a hormone sensitive cancer. As described above, the amount of microbial DNA may be determined by an amplification and/or high throughput sequencing technique. In some embodiments, the subject is administered a probiotic organism when there is a significantly decreased amount or level of bacterial DNA in the test sample when compared to a level of bacterial DNA in a control sample. In this case, the probiotic organism may be administered at a therapeutically effective dose. The method may optionally include a step of extracting a DNA sample from the test sample from the subject prior to amplifying the bacterial DNA sample.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

In some embodiments, the probiotic organism described above may be administered in combination with one or more additional therapeutic agents for the treatment of cancer. “In combination” or “in combination with,” as used herein, means in the course of treating the same cancer in the same subject using two or more agents, drugs, treatment regimens, treatment modalities or a combination thereof, in any order. This includes simultaneous administration, as well as in a temporally spaced order of up to several days apart. Such combination treatment may also include more than a single administration of any one or more of the agents, drugs, treatment regimens or treatment modalities. Further, the administration of the two or more agents, drugs, treatment regimens, treatment modalities or a combination thereof may be by the same or different routes of administration.

Examples of therapeutic agents that may be administered in combination with the probiotic organism include, but are not limited to, anti-cancer agents and radioisotopes. The therapeutic agent may also include a metal, metal alloy, intermetallic or core-shell nanoparticle bound to a chelator that acts as a radiosensitizer to render the targeted cells more sensitive to radiation therapy as compared to healthy cells.

In one embodiment, the therapeutic agent is an anti-cancer agent. Anti-cancer agents that may be used in accordance with the embodiments described herein are often cytotoxic or cytostatic in nature and may include, but are not limited to, alkylating agents; antimetabolites; anti-tumor antibiotics; topoisomerase inhibitors; mitotic inhibitors; hormones (e.g., corticosteroids); targeted therapeutics (e.g., selective estrogen receptor modulators (SERMs)); toxins; immune adjuvants, immunomodulators, and other immunotherapeutics (e.g., therapeutic antibodies and fragments thereof, recombinant cytokines and immunostimulatory molecules—synthetic or from whole microbes or microbial components); enzymes (e.g., enzymes to cleave prodrugs to a cytotoxic agent at the site of the tumor); nucleases; antisense oligonucleotides; nucleic acid molecules (e.g., mRNA molecules, cDNA molecules or RNAi molecules such as siRNA or shRNA); chelators; boron compounds; photoactive agents and dyes. Examples of anti-cancer agents that may be used as therapeutic agents in accordance with the embodiments of the disclosure include, but are not limited to, 13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bexarotene, bicalutamide, bortezomib, bleomycin, busulfan, calcium leucovorin, citrovorum factor, capecitabine, canertinib, carboplatin, carmustine, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, ozogamicin, goserelin, granulocyte—colony stimulating factor, granulocyte macrophage-colony stimulating factor, hexamethylmelamine, hydrocortisone hydroxyurea, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, PEG Interferon, pegaspargase, pegfilgrastim, PEG-L-asparaginase, pentostatin, plicamycin, prednisolone, prednisone, procarbazine, raloxifene, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tretinoin, trimitrexate, alrubicin, vincristine, vinblastine, vindestine, vinorelbine, vorinostat, or zoledronic acid.

Therapeutic antibodies and functional fragments thereof, that may be used as anti-cancer agents in accordance with the embodiments of the disclosure include, but are not limited to, alemtuzumab, bevacizumab, cetuximab, edrecolomab, gemtuzumab, ipilimumab, ibritumomab tiuxetan, panitumumab, rituximab, tositumomab, and trastuzumab, anti-PD1 antibodies and anti-PD1 ligand antibodies, and other antibodies associated with specific diseases listed herein.

Toxins that may be used as anti-cancer agents in accordance with the embodiments of the disclosure include, but are not limited to, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

Radioisotopes that may be used as therapeutic agents in accordance with the embodiments of the disclosure include, but are not limited to, ³²P, ⁸⁹Sr, ⁹⁰Y, ^{99m}Tc, ⁹⁹Mo, ¹³¹I, ¹⁵³Sm, ¹⁷⁷Lu, ¹⁸⁶Re, ²¹³Bi, ²²³Ra and ²²⁵Ac.

### Decreasing Levels of Steroid Hormones and Polycyclic Aromatic Hydrocarbons in Tissue to Prevent or Reduce the Risk of Cancer

Increased levels of steroid hormones known to cause hormone-sensitive cancer may be a risk factor that increases the risk of hormone-sensitive cancers. For example, women that are exposed to high levels of estrogen in the breast tissue may have an increased risk of breast cancer. Thus, decreasing the amount of estrogen in breast tissue may help prevent or reduce the risk of breast cancer. Additionally, polycyclic aromatic hydrocarbons (PAHs) include one or more fused aromatic rings and are chemical products of combustion from coal burners, fuel, cigarette smoke, and various other sources. PAHs have been shown to be carcinogenic and to increase the risk of breast cancer in a variety of ways. The most common PAHs are weakly estrogenic (estrogen mimicking), due to interactions with the cellular estrogen receptor (ER). Thus, as discussed above, decreasing the levels of PAHs in breast tissue may help prevent or reduce the risk of breast cancer.

Thus, some of the methods described herein are directed to decreasing the level of a steroid hormone in a subject to treat or prevent or reduce the risk of developing a steroid-hormone sensitive or dependent cancer (e.g., breast cancer). In such embodiments, the method may include a step of administering a therapeutically effective amount or dose of a probiotic organism to a subject. Examples of hormone-sensitive cancers include, but are not limited to, breast cancer, prostate cancer, ovarian cancer, endometrial cancer, testicular cancer, uterine cancer, and cervical cancer as described above. In one embodiment, the subject is at risk of having a hormone-sensitive cancer.

In some embodiments, the methods may be used to decrease levels of a steroid hormone that is known to play a role in the development of hormone-sensitive cancer. In one embodiment, the hormone-sensitive cancer is breast cancer and the steroid hormone molecule is an estrogen molecule. In one embodiment, the estrogen molecule may be estrone, estradiol, or estriol. In one aspect, the estrogen molecule is estradiol.

In certain embodiments, the levels of a steroid hormone may be decreased by administering a therapeutically effective dose of a probiotic organism at a dose sufficient to maintain a level of bacterial DNA in a test sample at a level that is approximately equal to or greater than a level of bacterial DNA in a control sample. The probiotic organism may be a bacterium that can degrade an organic molecule that has at least one carbon ring as described above and is also administered as described above.

According to other embodiments, methods of decreasing levels of a steroid hormone in a subject are provided. Such methods may include a step of amplifying a bacterial DNA sample in a test tissue sample from the subject to determine an amount of bacterial DNA. As described above, the amount of bacterial DNA may be determined by an amplification and/or high throughput sequencing technique. In some embodiments, the subject is administered a probiotic organism when there is a significantly decreased amount or level of bacterial DNA in the test sample when compared to a level of bacterial DNA in a control sample. In this case, the probiotic organism may be administered at a dosage sufficient to maintain a bacterial DNA level in the test sample at a level that is approximately equal to a level of bacterial DNA in a control sample. The method may optionally include a step of extracting a DNA sample from the test tissue sample from the subject prior to amplifying the bacterial DNA sample.

In other embodiments, the level maintained is greater than a level of bacterial DNA in the control sample. The therapeutically effective dose of the probiotic organism is administered as described above.

The methods as described herein are also directed to decreasing the level of polycyclic aromatic hydrocarbons (PAHs) in a tissue to prevent or reduce the risk of breast cancer. These methods include administering to the subject a therapeutically effective dose of a probiotic organism that includes one or more bacterial strains that degrade organic molecules that have at least one carbon ring. In one embodiment, the organic molecule that includes at least one carbon ring is a PAH.

### Stimulating an Immune Response

Natural killer T (NKT) cells play a role in the regulation of inflammatory immune responses. A subset of NKT cells, called invariant NKT (iNKT) cells, express both natural killer cell surface markers and highly restricted T-cell receptors (TCRs). These cells possess properties of both innate and adaptive immune cells. Similar to cells of the innate immune system, iNKT cells interact with a limited subset of antigens and fail to develop immunological memory; however, they also produce large amounts of cytokines that stimulate and modulate an adaptive immune response. iNKT cells have been implicated in infectious disease, allergy, autoimmunity, and tumor surveillance. They have been shown to promote cell-mediated immunity to tumors and infectious organisms, including bacteria and viruses, and to suppress the cell-mediated immunity associated with autoimmune diseases and allograft rejection. Thus, stimulating an increased immune response through activation of iNKT cells would be beneficial for both prevention and treatment of inflammation and cancer.

The iNKT cell TCR recognizes self and foreign glycolipid antigens bound to, or presented by, CD1d proteins on antigen presenting cells (APCs). CD1d APCs include monocytes, dendritic cells, and B cells. Certain genera of bacteria contain glycosphingolipids, which are a type of glycolipid, in their cell membranes. iNKT cells have been shown to recognize, and be activated by, CD1d-presented glycosphingolipids produced by different genera of bacteria, including Sphingomonas.

Unexpectedly, as described in the examples in more detail below, normal breast tissue containing no tumor cells was significantly enriched in the bacteria Sphingomonas yanoikuyae compared to ER+ breast cancer tumor tissue. Additionally, levels of antibacterial response genes were shown to be down-regulated in breast cancer tissues compared to normal adjacent breast tissue. Therefore, tumor tissue having a lower level of bacteria that contain glycosphingolipids may have a reduced immune response compared with normal tissue that has enriched levels of these bacteria. As a result, activation of iNKT cells in inflamed or tumor tissue by bacteria containing glycosphingolipids may stimulate an increased immune response which would be a beneficial immune therapy for patients suffering from diseases related to inflammation and cancer.

Accordingly, methods as described herein are directed to stimulating an increased immune response in a diseased tissue by administering a therapeutically effective dose of a probiotic organism and/or functional components of the organism (e.g., antigens or protein fragments of the organism; or ligands, or secreted proteins that are isolated from the probiotic organism) to a subject containing the diseased tissue. In some embodiments, the therapeutically effective dose may be a dose as described above. For example, the therapeutically effective dose is sufficient to maintain a bacterial DNA level in the diseased tissue at a level that is approximately equal to a level of bacterial DNA in a control sample. In other examples, the therapeutically effective dose is sufficient to increase the bacterial load in the diseased tissue, while the bacterial load in the control sample remains approximately the same. In other examples, the therapeutically effective dose is sufficient to maintain a bacterial DNA level in the diseased tissue at a level that is greater than a level of bacterial DNA in a control sample.

In certain embodiments, the probiotic organism may include bacteria that contain ligands that are recognized by and which activate NKT cells. In some embodiments, the NKT cells are iNKT cells. In other embodiments, the ligands are glycosphingolipid antigens contained in the cell membrane of certain bacteria. Bacteria that have been shown to contain glycosphingolipids that activate iNKT cells include genera such as Sphingomonas and Borrelia. Streptococcus pneumoniae and group B Streptococcus are examples of lethal bacterial pathogens that also activate iNKT cells. In one embodiment, the bacterium that stimulates an increased immune response through activation of iNKT cells is Sphingomonas yanoikuyae. Sphingomonas yanoikuyae is a species of bacteria that is not highly virulent and would therefore be an exemplary probiotic organism for treatment purposes.

In one embodiment, the bacterial DNA level in the diseased tissue is maintained at a level that is approximately equal to or greater than a level of bacterial DNA in a control sample. The levels of bacterial DNA may be quantified as described above to determine the levels found in the diseased tissue and the control sample.

Additionally, in some embodiments, a method of stimulating an increased immune response in a subject containing a diseased tissue is provided. Such methods may include a step of amplifying or otherwise detecting a bacterial DNA sample in a test tissue sample from the subject and determining an amount of bacterial DNA. As described above, the amount of bacterial DNA may be determined by an amplification and/or high throughput sequencing technique. In some embodiments, the subject is administered a probiotic organism and/or functional components of the organism when there is a significantly decreased amount or level of bacterial DNA in the test sample when compared to a level of bacterial DNA in a control sample. In this case, the probiotic organism may be administered at a dosage sufficient to maintain a bacterial DNA level in the test sample at a level that is approximately equal to a level of bacterial DNA in a control sample. The method may optionally include a step of extracting a DNA sample from the test tissue sample from the subject prior to amplifying the bacterial DNA sample.

The levels of bacterial DNA may be determined and amplified as described herein.

In some embodiments, the diseased tissue may include any tissue that is inflamed or cancerous. In one embodiment, the diseased tissue is a tissue containing tumor cells such as a breast cancer tissue. In other embodiments, a diseased tissue is one that is inflamed.

As described herein, the probiotic organism that has the ability to activate NKT cells or other antitumor responsive immune cells may be administered, by any suitable route of administration, alone or as part of a pharmaceutical composition as described in detail above. Additionally, the therapeutically effective amount of probiotic organism may be administered in an amount as described above.

According to some embodiments, the probiotic organism described above may be administered in combination with a therapeutically effective amount of one or more immunologic agents to further stimulate the immune system. There are two main types of immunologic agents, active and passive. Active immunologic agents, such as vaccines, stimulate an immune response to one or more specific antigenic types. In contrast, passive immunologic agents do not have antigenic specificity but can act as general stimulants that enhance the function of certain types of immune cells. Immunologic agents that may be used in combination with the probiotic organism include, but are not limited to, immunostimulant substances that modulate the immune system by stimulating the function of one or more of the system's components.

In some embodiments, immunologic agents that may be used in accordance with the methods described herein include, but are not limited to, vitamins, minerals, nutrients, herbs, plant-derived substances, fungi, animal or insect-derived substances, adjuvants, antioxidants, amino acids, cytokines, chemokines, hormones, T cell costimulatory molecules, general immune-stimulating peptides, gene therapy, immune cell-derived therapy, and therapeutic antibodies.

In some embodiments, the one or more immunologic agents may include, but are not limited to, vitamin C, vitamin A, vitamin E, vitamin B-6), carotenoids and beta carotene, selenium, zinc, flavanoids and bioflavanoids, iron chelators, astragalus, beta-glucans, echinacea, elderberry, garlic, ginger, ginseng, Ganoderma lucidum (Reishi or Ling Zhi), medicinal mushrooms (Reishi or Agaricus blazei), bee propolis, snake venom, scorpion, colostrum (e.g., bovine colostrum), indirubin, cordycepssinensis, scutellaria Baicalensis georgi, Rhemannia glutinosa (Chinese Foxglove, Shen di Huang), quercetin, coenzyme Q10, lysine camitine, glutathione-containing compounds, omega-3 fatty acids, prolactin, growth hormone, alpha-lipoic acid, lentinan, polysaccharide-K (MC-S), synthetic cytosine phosphate-guanosine (CpG), oligodeoxynucleotides, interleukins (e.g., IL-2 or IL-12), tumor necrosis factor alpha or beta (TNF-α or -β), granulocyte colony-stimulating factor (G-CSF), B7-1, ICAM-1, LFA-3, proline-rich polypeptides (PRPs, which can be made or derived from mammalian cololstrum such as bovine colostrum), imiquimod, beta-glucans, BCG vaccine, tumor antigens, killed tumor cell therapy, gene therapy vectors expressing cytokines, T cell costimulatory molecules or other suitable immunostimulatory molecules, dendritic cell based immunotherapeutics, T cell based adoptive immunotherapeutics.

In other embodiments, the one or more immunologic agent used in the methods described herein may be a therapeutic antibody or a functional fragment thereof that targets cancer cells. Passive immunotherapy in the form of therapeutic antibodies has been the subject of considerable research and development as anti-cancer agents. Therapeutic antibodies are typically administered in maximum tolerated doses to block target receptors that are overexpressed on cancer cells, blocking the receptor's function systemically. However, given at a dose that is substantially lower than the maximum tolerated dose (e.g., ½ to 1/1000th of the standard dose) allows the therapeutic antibody to act as an immunostimulant. After binding a target cancer cell, therapeutic antibodies or functional fragments thereof may stimulate cytotoxic immune-mediated responses, such as antibody-dependent cell-mediated cytotoxicity and complement-dependent cytotoxicity, mediated by Fc region activation of complement or Fc receptor (FcR) engagement. After cancer cells have been lysed, macrophages and other phagocytic, antigen presenting immune cells may engulf the components of the lysed cell and present cancer cell antigens to stimulate an acquired immune response against the cancer cells.

Examples of therapeutic antibodies that may be used as an immunologic agent according to the embodiments of the disclosure include, but are not limited to, alemtuzumab, bevacizumab, cetuximab, edrecolomab, gemtuzumab, ibritumomab tiuxetan, ipilimumab, panitumumab, rituximab, tositumomab, and trastuzumab.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1: Healthy Breast Tissue Exhibits Significantly Higher Levels of Bacterial DNA Compared with Tumor Breast Tissue

Breast cancer affects one in eight women in their lifetime. Though diet, age and genetic predisposition are established risk factors, the majority of breast cancers have unknown etiology. The human microbiota refers to the collection of microbes inhabiting the human body. Imbalance in microbial communities, or microbial dysbiosis, has been implicated in various human diseases including obesity, diabetes, and colon cancer. As provided in Examples 1 and 2 below, the role of microbiota in breast cancer was investigated in breast tumor tissue and paired normal adjacent tissue from the same patient using next-generation sequencing. In a qualitative survey of the breast microbiota DNA, it was shown that the bacterium Methylobacterium radiotolerans is relatively enriched in tumor tissue, while the bacterium Sphingomonas yanoikuyae is relatively enriched in paired normal tissue. The relative abundances of these two bacterial species were inversely correlated in paired normal breast tissue but not in tumor tissue, indicating that dysbiosis is associated with breast cancer. Furthermore, the total bacterial DNA load was reduced in tumor versus paired normal and healthy breast tissue as determined by quantitative PCR. Interestingly, bacterial DNA load correlated inversely with advanced disease, a finding that could have broad implications in diagnosis and staging of breast cancer. Lastly, lower basal levels of antibacterial response gene expression were observed in tumor versus healthy breast tissue. Taken together, these data indicate that microbial DNA is present in the breast and that bacteria or their components may influence the local immune microenvironment. These findings suggest a previously unrecognized link between dysbiosis and breast cancer which has potential diagnostic and therapeutic implications.

As described in this and Example 2 below, healthy breast tissue was shown to exhibit significantly higher levels of bacteria compared to tissues obtained from estrogen receptor sensitive tumor and estrogen receptor negative tumor breast tissue. Additionally, although the overall composition of the breast microbiota was not significantly altered in healthy breast tissue versus tumor breast tissue, the level of bacteria was significantly increased in healthy tissue.

Materials and Methods

Breast tissue specimens. Formalin fixed paraffin-embedded (FFPE) tumor and matched healthy tissues were obtained from Saint John's Health Center in accordance with institutional IRB requirements approved by the Saint John's Health Center/John Wayne Cancer Institute joint institutional review board and Western institutional review board (Western IRB). Written consent was specifically waived by the approving IRB.

Fluorescence in-situ hybridization (FISH). 4 μm tissue sections were affixed to glass slides. FISH was performed on serial sections of FFPE tissues using the bacterial 16S rDNA probe EUB338. The probe NONEUB338 was used as a control. The staining protocol was adopted from Klitgaard et al. with slight modifications (Klitgaard, 2005). Briefly, 5 ng/ul of biotinylated probe was hybridized to tissues for 16 h in a humidified 37° C. incubator. Probes were detected using Streptavidin-Alexa568 conjugate (Invitrogen). Images were acquired using a Leica LMD7000 microscope.

Quantitative PCR (qPCR) for bacterial copy numbers. Total genomic DNA (gDNA) was extracted from FFPE tissues using QIAamp DNA FFPE Tissue kit per manufacturer's instructions with slight modifications. Purified gDNA was eluted twice from the column using ultrapure water. All extractions were performed in a designated clean (pre-PCR) room.

qPCR was performed using universal bacterial rDNA primers 63F (forward, 5′-GCA GGC CTA ACA CAT GCA AGT C-3′) and 355R (reverse, 5′-CTG CTG CCT CCC GTA GGA GT-3′) on microbial DNA extracted from FFPE tissue. Bacterial copy numbers were normalized by the total amount (μg) of extracted DNA quantified using Quanti-it PicoGreen dsDNA Reagent Kit (Invitrogen). Samples were randomized and processed in a blinded manner. The genus-specific primers Sph-spt 694F (forward, 5′-GAG ATC GTC CGC TTC CGC-3′) and Sph-spt 983R (reverse, 5′-CCG ACC GAT TTG GAG AAG-3′) were used to quantify Sphingomonas (Lin, 2011). The species-specific primers 5F (forward, 5′-CTT GAG TAT GGT AGA GGT T-3′) and 8R (reverse, 5′-CAA ATC TCT CTG GGT AAC A-3′) were used to quantify M. radiotolerans (Nishio, 1997).

Results

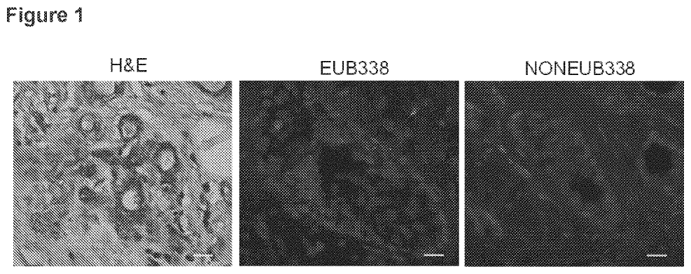

Bacteria are present in the breast ducts of women with breast cancer. To determine the histological location of microbial communities in the breast, fluorescence in-situ hybridization (FISH) using a probe specific for bacterial 16S rDNA (EUB338) was performed on breast tumor tissue. It was found that bacteria were clustered around breast ducts in both tumor and matched normal tissues (FIG. 1). Because the majority of breast cancers arise from the breast ductal epithelium, it is likely that the breast microbiota may influence breast cancer development and/or progression. Thus, the microbial communities in the breast were further characterized.

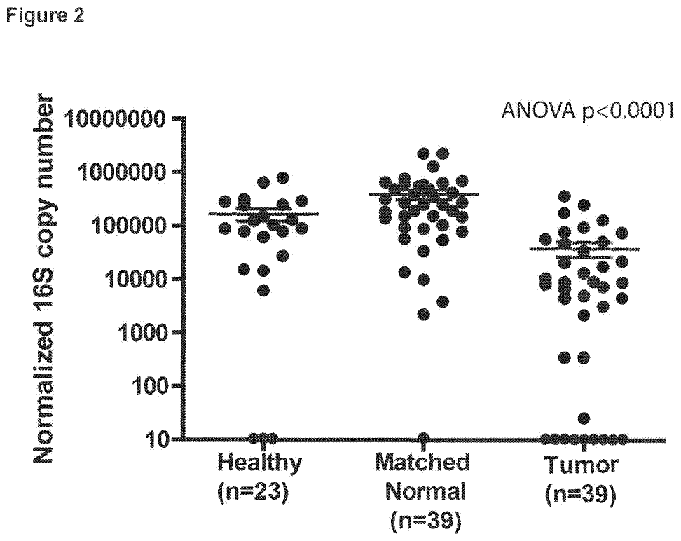

Matched normal tissue contains significantly higher amounts of bacteria compared to tumor tissue. To determine if there was a quantitative difference in microbiota or bacterial load in matched normal tissue versus tumor tissue, microbial DNA was extracted from formalin fixed paraffin-embedded tissue blocks and quantified by quantitative PCR (qPCR) analysis to enumerate 16S ribosomal DNA (rDNA) copy numbers as a surrogate measure of total bacterial counts (Castillo, 2006). Quantitative PCR performed using universal bacterial rDNA primers 63F and 355R revealed significantly higher (˜10-fold) copy numbers of 16S rDNA in matched healthy tissue (391,096±81,570) compared to tumor tissue (37,582±11,783) using Kruskal-Wallis nonparametric ANOVA with Dunn's multiple comparison post-test to account for uneven sample numbers between the three groups studied (healthy vs. tumor p<0.01, paired normal vs. tumor p<0.001, healthy vs. paired normal n.s., FIGS. 2 and 22A). Bacterial levels in paired normal tissue, on the other hand, did not differ significantly from those found in healthy breast tissue (164,484±42,477) (mean s.e.m.) using Kruskal-Wallis nonparametric ANOVA with Dunn's Multiple Comparison post-test

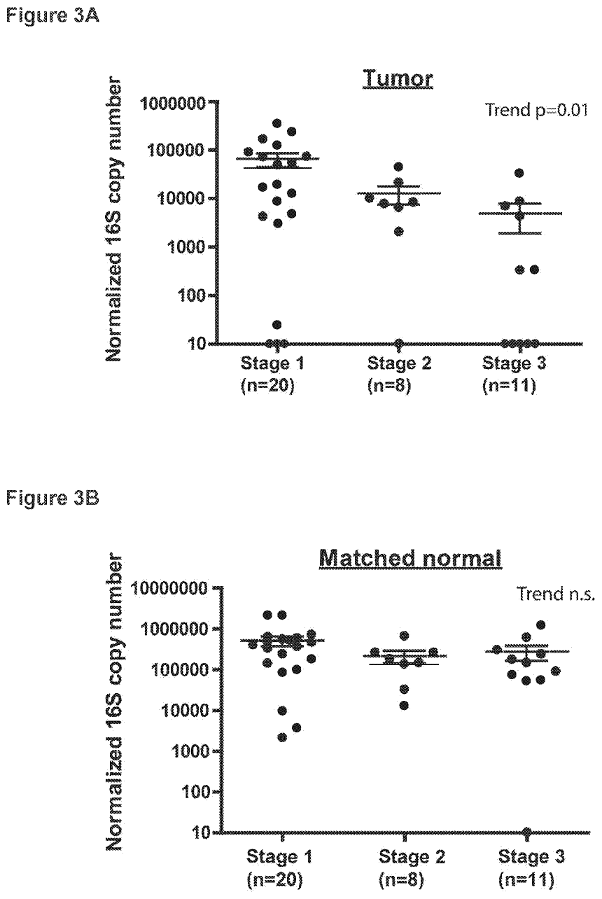

Moreover, an inverse correlation between breast cancer stage and bacterial load in tumor tissue, but not in paired normal tissue, was observed using Cuzick's Trend test analysis (FIGS. 3, 22B and 22C). Tumors from Stage 1 patients had the highest copy numbers of bacterial DNA (69,489±23,382) (mean±s.e.m.), followed by Stage 2 patients (16,867±6,152), with Stage 3 patients having the lowest bacterial load amongst the three groups (5,258±2,758) (Trend p=0.0056) (FIG. 22B). In contrast, paired normal tissue from the same patients did not have different bacterial copy numbers (Trend p=0.1702) (FIG. 22C). These data suggest an inverse correlation between severity of disease and bacterial load at the tumor site, which may have diagnostic implications in breast cancer.

### Example 2: Healthy Breast Tissue Exhibits Significantly Higher Levels of Bacteria that can Degrade Aromatic Molecules and Activate NKT Cells Compared with Tumor Breast Tissue

The data set forth in Example 1 led to further investigation of the composition of the microbiota in healthy and tumor breast tissues. As discussed in this Example, the species of bacteria known to degrade aromatic molecules was significantly enriched in healthy breast tissue compared with estrogen receptor positive (ER+) tumor breast tissue. Additionally, these bacteria have been shown to produce a ligand that activates invariant natural killer T (iNKT) cells, which are known to be important for immune responses to autoimmune diseases, cancer, inflammation, and infection. Levels of expression of antibacterial genes were shown to be down-regulated in breast cancer tissue compared to normal adjacent breast tissue, which may be due to a reduced activation of NKT cells or other immune cells in breast cancer tissue.

Materials and Methods

In addition to those described in Example 1 above, the following materials and methods were used.

16S microbial DNA pyrosequencing. The microbiome in breast cancer was the initial target of investigation and ER+ tumors were chosen for study. Due to the variability of the microbiome from individual to individual, it was decided that matched tissue (paired normal and tumor) from the same individual would provide the best comparison of microbial communities. Twenty paraffin-embedded paired samples were used for this purpose. Total genomic DNA was extracted from samples using the QIAamp DNA FFPE Tissue kit (Qiagen) per manufacturer's instructions. The genomic DNA (gDNA) (from Subjects 1-20) was submitted to Second Genome Inc., for pyrosequencing and analysis. The gDNA was amplified using fusion primers targeting the bacterial 16S V4 rDNA with indexing barcodes. All samples were amplified with two differently barcoded V4 fusion primers and pooled for sequencing on the Illumina Miseq with 150 bp paired-end reads. 60,248±14,229 (mean±s.d.) reads were obtained per sample.

Data analysis for pyrosequencing. Sequences were quality filtered and demultiplexed using QIIME (Caporaso, 2010) and custom scripts with exact matches to the supplied DNA barcodes. Resulting sequences were then searched against the Greengenes reference database of 16S sequences (DeSantis, 2006) and clustered at 97% by uclust (Edgar, 2010). The longest sequence from each Operation Taxonomic Unit (OTU) was used as the OTU representative sequence and assigned taxonomic classification via Mothur's Bayesian classifier (Schloss, 2009) and trained against the Greengenes database clustered at 98%. To account for biases caused by uneven sequencing depth, an equal number of random sequences were selected from each sample prior to calculating community-wide dissimilarity measures. The sequence data has been submitted to the European Nucleotide Archive, PRJEB4755.

Quantitative PCR (qPCR) for bactenal copy numbers. As described above, qPCR was performed using universal bacterial rDNA primers 63F (forward, 5′-GCA GGC CTA ACA CAT GCA AGT C-3′) and 355R (reverse, 5′-CTG CTG CCT CCC GTA GGA GT-3′) on microbial DNA extracted from FFPE tissue. All samples from pyrosequencing were also assessed for bacterial copy number (Subjects 1-20, excluding Subjects 3 and 5 due to limited material) and additional paraffin-embedded tissue specimens (from patients with breast cancer-subjects 21-41) were obtained at a later time after the initial pyrosequencing experiment, and thus were used only in the quantification experiments as previously described (Castillo, 2006) to enumerate the amount of total bacteria. DNA from healthy specimens was obtained from patients undergoing reduction mammoplasty, with no evidence of breast cancer. Bacterial copy numbers were normalized by the total amount (μg) of extracted DNA quantified using Quanti-it PicoGreen dsDNA Reagent Kit (Invitrogen). Samples were randomized and processed in a blinded manner. The genus-specific primers Sph-spt 694F (forward, 5′-GAG ATC GTC CGC TTC CGC-3′) and Sph-spt 983R (reverse, 5′-CCG ACC GAT TTG GAG AAG-3′) were used to quantify Sphingomonas (Lin, 2011). The species-specific primers 5F (forward, 5′-CTT GAG TAT GGT AGA GGT T-3′) and 8R (reverse, 5′-CAA ATC TCT CTG GGT AAC A-3′) were used to quantify M. radiotolerans (Nishio, 1997) (Subjects 1-20).

PCR array of expression of antibacterial response genes. Given the superior quality of mRNA from fresh-frozen tissue, fresh-frozen tissue was used rather than formalin fixed, paraffin embedded tissue in the gene expression study. RNA was extracted from fresh-frozen breast tissue from three healthy reduction mammoplasty patients and from tumor tissue of six patients with breast cancer (Subjects 42-47), then converted to cDNA using iScript cDNA synthesis kit (Biorad). cDNA was added to Human Antibacterial Response PCR Arrays (Qiagen) and the arrays were processed according to manufacturer's instructions. Data were analyzed using RT² Profiler PCR Array Data Analysis Software version 3.5, using beta-actin gene expression for normalization.

Statistical analysis. Student's t tests, Kruskal-Wallis nonparametric ANOVA tests and Spearman correlation tests were performed using Graphpad Prism software (Graphpad). Cuzick's Trend tests were performed using StatsDirect statistical software (StatsDirect). p<0.05 was used as the cut-off value for statistical significance.

Results

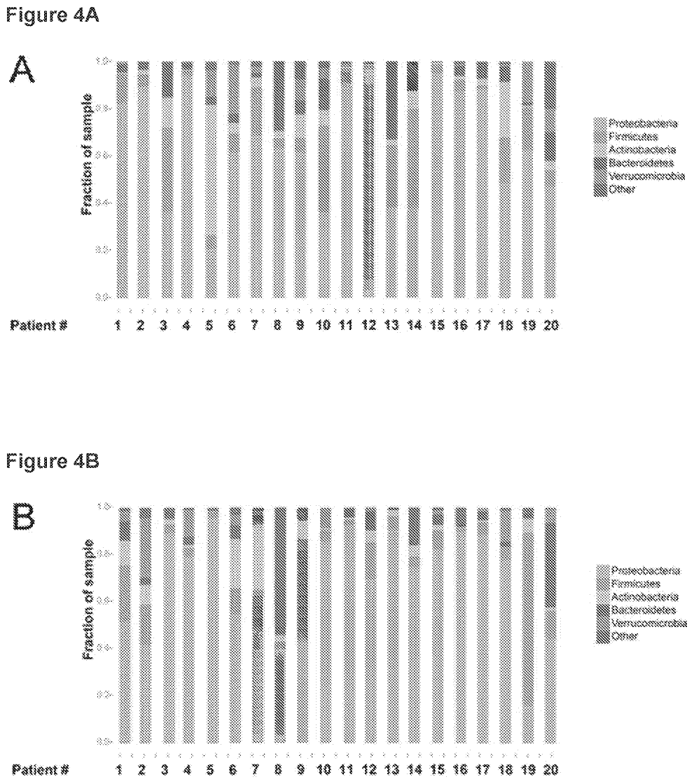

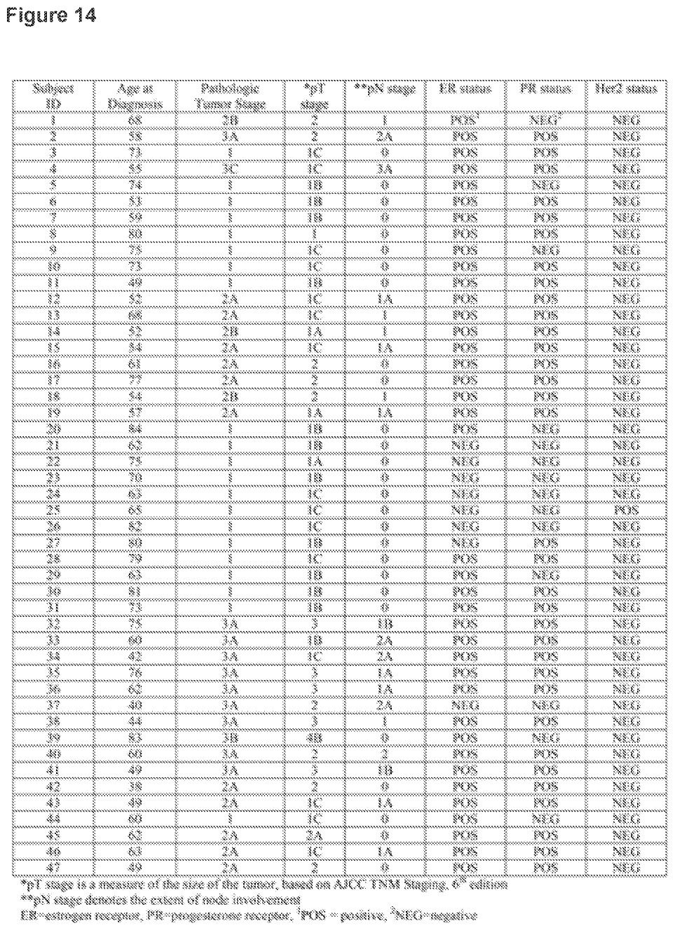

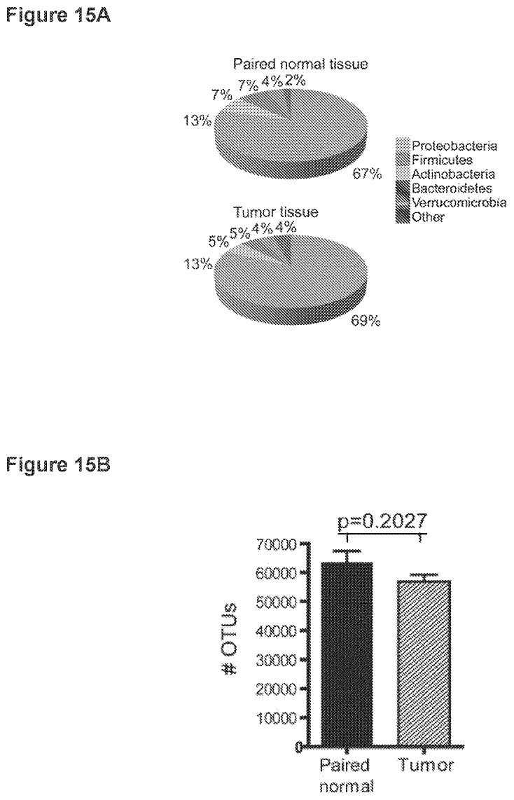

Shifts in the breast microbiota in matched normal tissue. The breast cancer microbiome has thus far not been described. The breast microbiota was surveyed in paired normal adjacent tissue (“paired normal”) and tumor tissue from 20 patients with estrogen receptor (ER)-positive breast cancer (clinical data reported in FIG. 14) using 16S pyrosequencing. The overall composition of the breast microbiota was not significantly altered in matched healthy tissue versus tumor tissue. The five richest phyla were Proteobacteria, Firmicutes, Actinobacteria, Bacteroidetes and Verrucomicrobia across all samples, accounting for an average of 96.6% of all sequences across samples, regardless of health status (FIGS. 4A and 4B; FIG. 15A, also see Example 2).

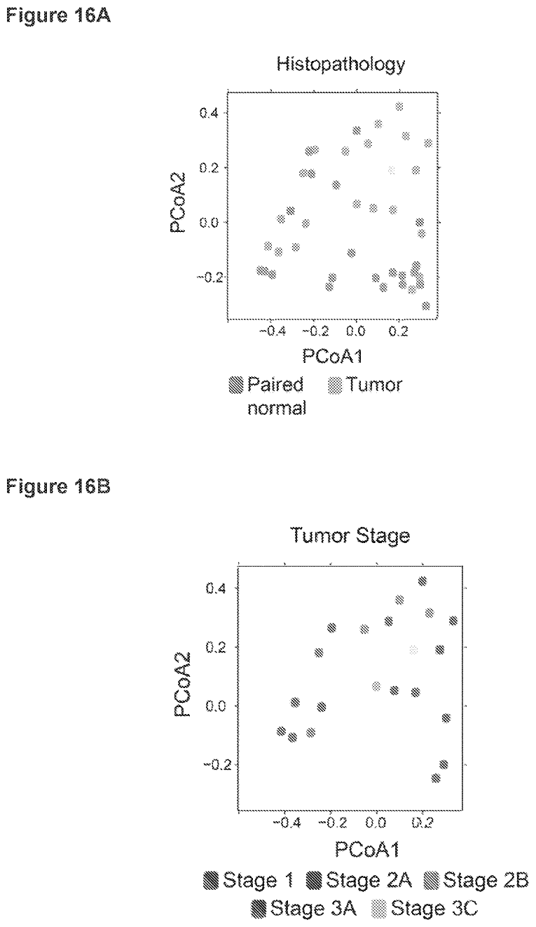

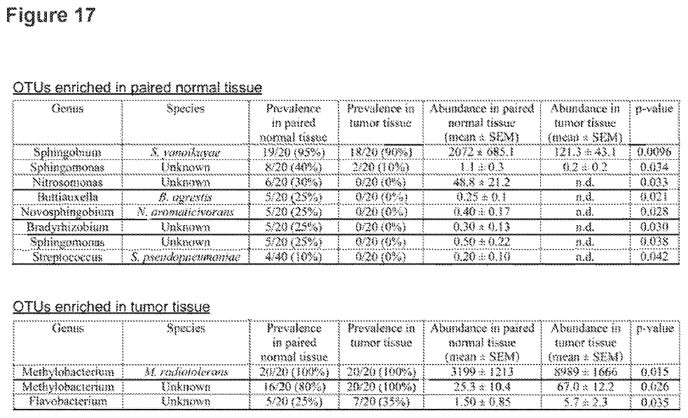

Sphingomonas yanoikuyae and Methylobacterium radiotolerans are significantly enriched. Based on a principle coordinates analysis (PCoA), no clustering was observed on the basis of health status, or other clinical variables including age, tumor staging and histological categories (FIGS. 16A and B). The number of operational taxonomic units (OTUs) detected did not vary between paired normal and tumor tissue, indicating that there was no significant difference in richness between the sampled communities (FIG. 15B). However, the abundance levels of the microbiota present in matched healthy tissue were significantly different than those found in tumor tissue as determined by Adonis testing (p=0.01). Of the 1614 OTUs detected, 11 OTUs were differentially abundant (p<0.05, FIG. 17).

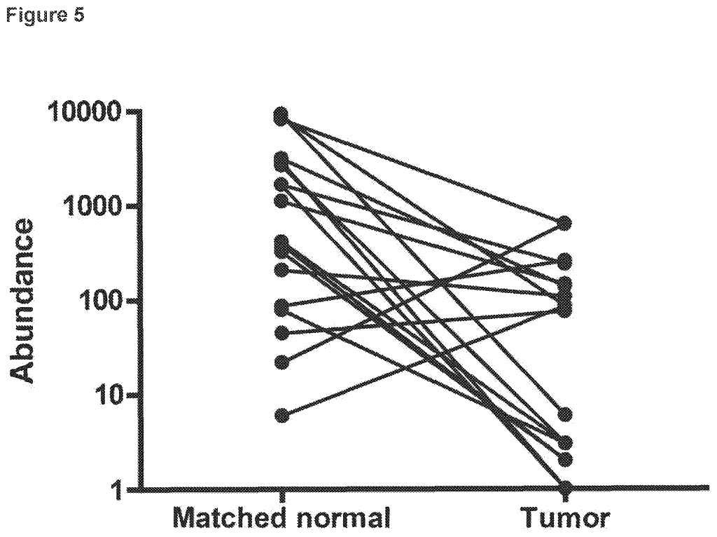

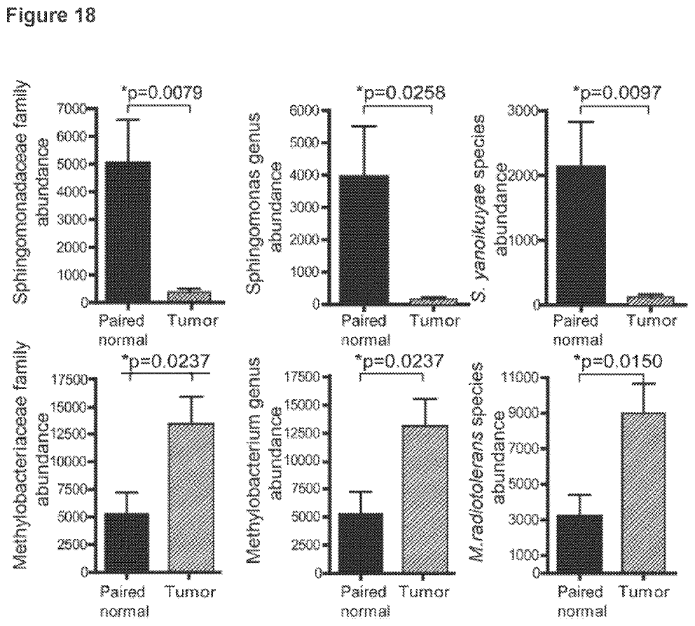

Of the 11 OTUs found to be differentially abundant, eight were more abundant in paired normal tissue and three were more abundant in tumor tissue. 50% (4/8) of the OTUs identified as more abundant in paired normal tissue belonged to the genus Sphingomonas (two from the genus Sphingomonas, one from the genus Sphingobium and one from the genus Novosphingobium) and 66.7% (2/3) of the OTUs identified as more abundant in tumor tissue belonged to the genus Methylobacterium (FIG. 17). The bacterium Sphingomonas yanoikuyae (S. yanoikuyae) was the most significantly enriched in matched normal tissue compared to tumor tissue (p=0.009, FIG. 5; p=0.0097, FIG. 18, top right panel). S. yanoikuyae was also found to be the most prevalent in paired normal tissue (FIG. 17). Detectable levels were found in 95% of healthy tissues and 60% of tumor tissues, with 15 out of 20 matched normal tissues having higher levels of the organism versus tumor tissue.

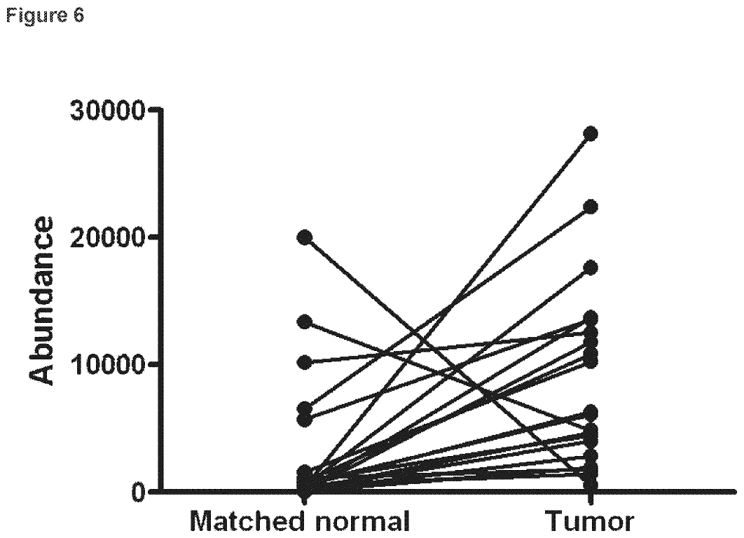

The bacterium Methylobacterium radiotolerans was significantly increased in tumor tissue compared to matched normal adjacent tissue (p=0.01; FIG. 6). The bacterium Methylobacterium radiotolerans (M. radiotolerans) was the most significantly enriched (p=0.0150, FIG. 18, bottom right panel) and the most prevalent (found in 100% of samples) in tumor tissue.

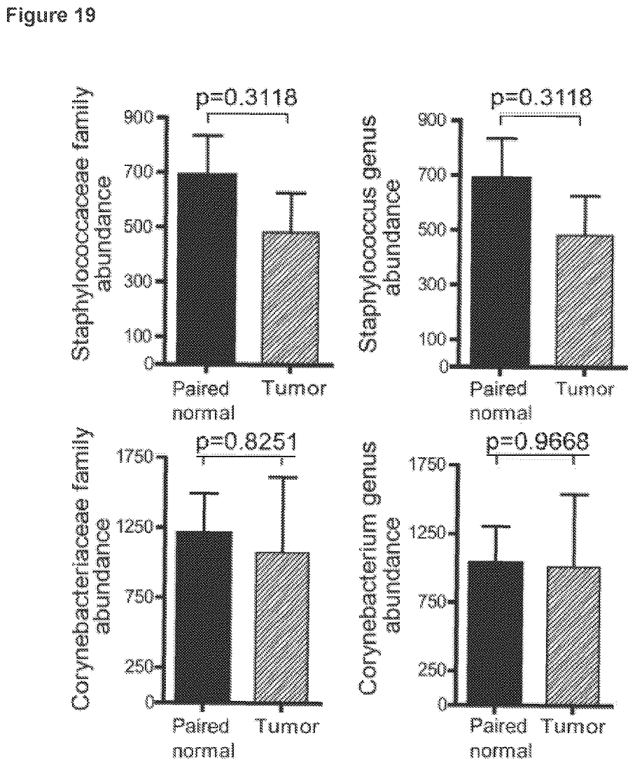

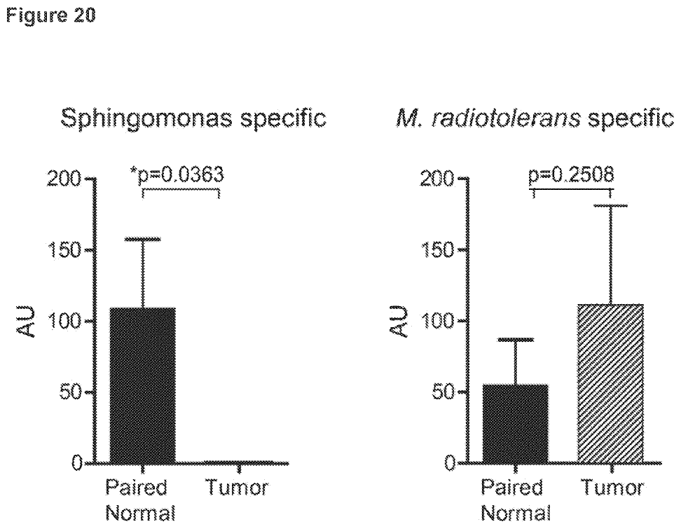

In contrast, the relative abundances of common skin bacteria including Staphylococcus and Corynebacterium did not vary significantly between paired normal and tumor tissue (FIG. 19, top panels compared with bottom panels, respectively). Since pyrosequencing provides a qualitative survey of relative abundances of microbiota, qPCR was used to determine if there was a quantitative difference in the levels of S. yanoikuyae and M. radiotolerans in paired normal and tumor tissue. Sphingomonas was detected in 40% of paired normal tissue and none of the corresponding tumor tissue, with absolute levels of Sphingomonas being significantly higher in paired normal tissue (p=0.0363, FIG. 20, left panel). In contrast, though M. radiotolerans was detected in all samples by qPCR, its absolute levels did not vary significantly between paired normal and tumor tissue (p=0.2508, FIG. 20, right panel), indicating that its higher relative abundance in tumor tissue reflects a decrease in other bacteria present and not an increase in the absolute level of the organism.

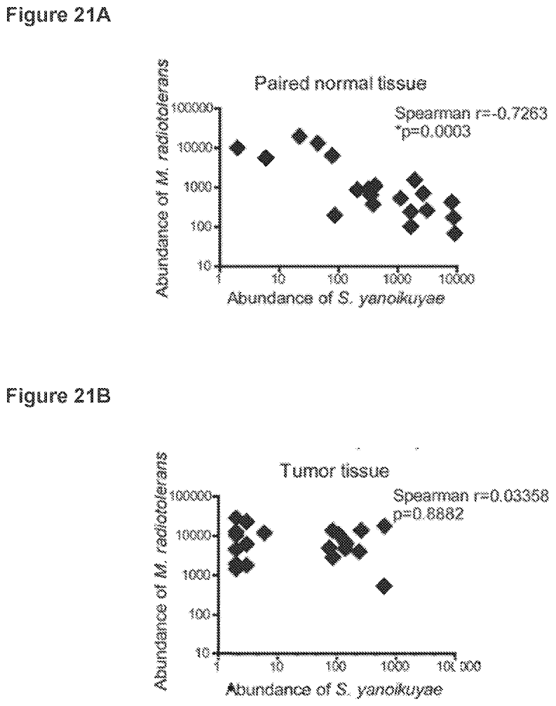

Notably, there was a strong inverse correlation between the abundance of S. yanoikuyae and M. radiotolerans in paired normal tissue (FIG. 21A, p=0.0003) which was not found in the corresponding tumor tissue (FIG. 21B). These data suggest that in paired normal tissue, S. yanoikuyae and M. radiotolerans may occupy similar niches and thus counterbalance each other in abundance. Meanwhile in tumor tissue, the quantity of S. yanoikuyae becomes significantly lower as the quantity of M. radiotolerans remains constant.

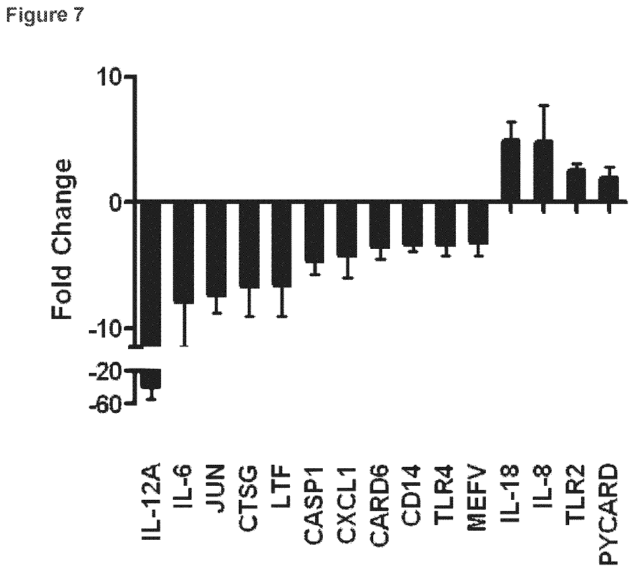

Antibacterial response genes are down-regulated in breast cancer tissues. The decreased bacterial load measured in tumor tissue compared with paired normal tissue and healthy tissue may influence the expression of antibacterial response genes in the tumor microenvironment. The levels of expression of antibacterial genes were down-regulated in breast cancer tissues compared to healthy adjacent breast tissue from a cancer patient (FIG. 7). Notably, IL-12A, a subunit of IL-12, was downregulated by 12 to 123-fold among samples (FIG. 7).

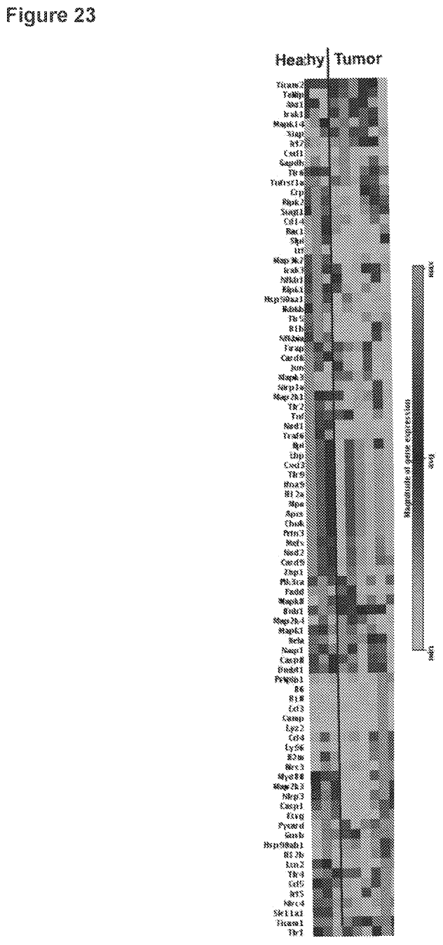

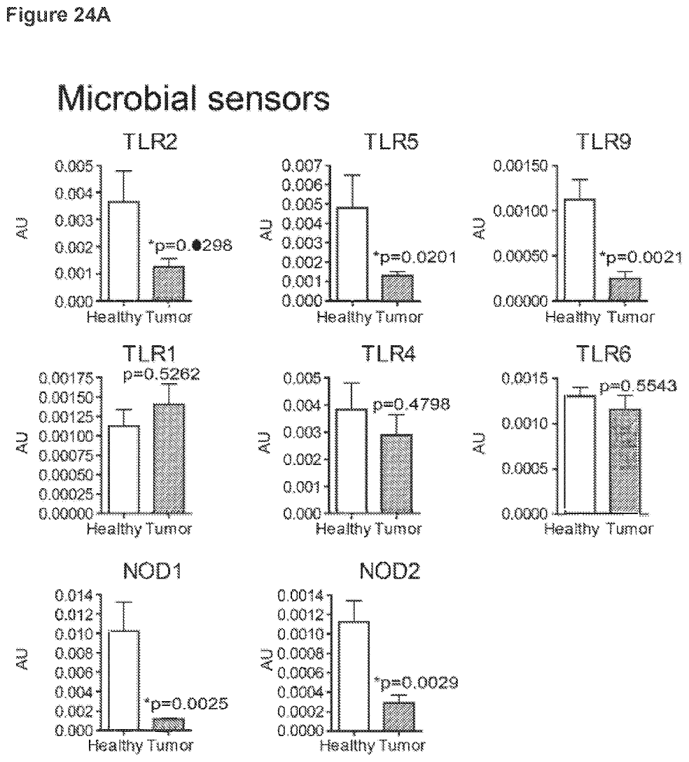

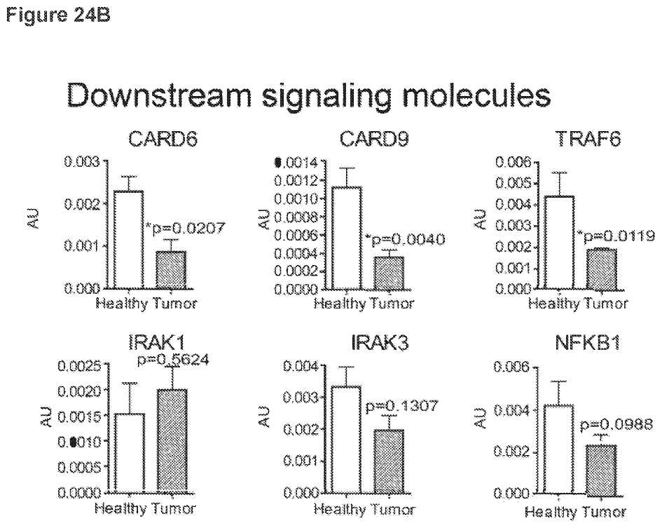

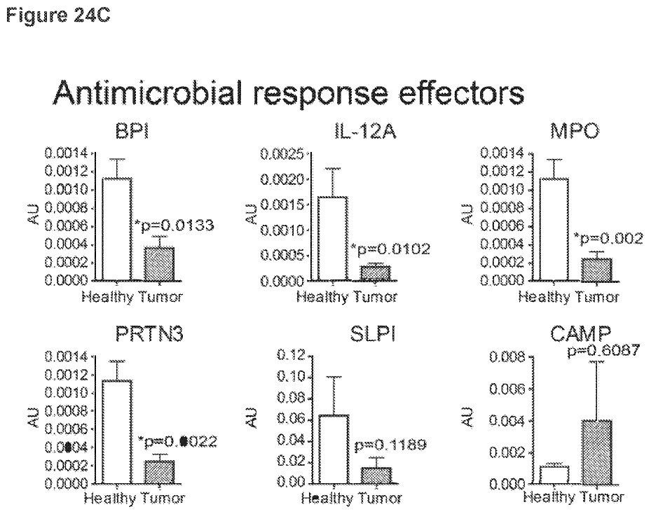

Further, gene expression profiles in breast tissue from three healthy patients undergoing reduction mammoplasty were compared with six patients with breast cancer (tumor tissue was used, clinical data reported in FIG. 14) using a targeted gene array for human antibacterial response genes normalized to the housekeeping gene beta-actin. One-third (28/84) of antibacterial genes surveyed were downregulated in tumor tissue, while the remaining two-thirds (56/84) were not significantly different between tumor and healthy tissue. Strikingly, none of the antibacterial genes surveyed were significantly upregulated in tumor tissue. The samples segregated into their tissue type, tumor vs. healthy by non-supervised hierarchical clustering, and a subset of genes were comparatively decreased in expression in tumor tissue compared with healthy tissue (FIG. 23). Of these genes, the transcripts of microbial sensors Toll-like receptors 2, 5 and 9 (TLR2, TLR5 and TLR9) were significantly reduced in tumor tissue (p=0.0298, p=0.0201 and p=0.0021, respectively), while expression levels of Toll-like receptors 1, 4 and 6 (TLR1, TLR4 and TLR6) were similar in healthy and tumor tissue (FIG. 24A). S. yanoikuyae is a species of Gram-negative bacteria that does not contain lipopolysaccharide (LPS) and therefore does not elicit TLR4-mediated responses (Kinjo, 2005). The cytoplasmic microbial sensors NOD receptors 1 and 2 (NOD1 and NOD2) were also expressed at lower levels in tumor tissues (p=0.0025 and p=0.0029, respectively), along with downstream signaling molecules for innate microbial sensors including CARD6, CARD9 and TRAF6 (p=0.0207, p=0.0040 and p=0.0119, respectively) (FIG. 24B). In addition, transcripts of antimicrobial response effectors were less abundant in tumor tissue, with BPI, MPO and PRTN3 levels being significantly lower compared with those found in healthy tissue (p=0.0133, p=0.002 and p=0.0022, respectively) (FIG. 24C). These data show a significant reduction in antibacterial responses in breast cancer tumor tissue.

T cell isolation from breast tissue. T cells were isolated from normal tissue taken from a reduction mammoplasty procedure using a previously established protocol. The T cells were cultured in the presence of IL-2 and stimulated with CD3/CD28 beads where indicated.

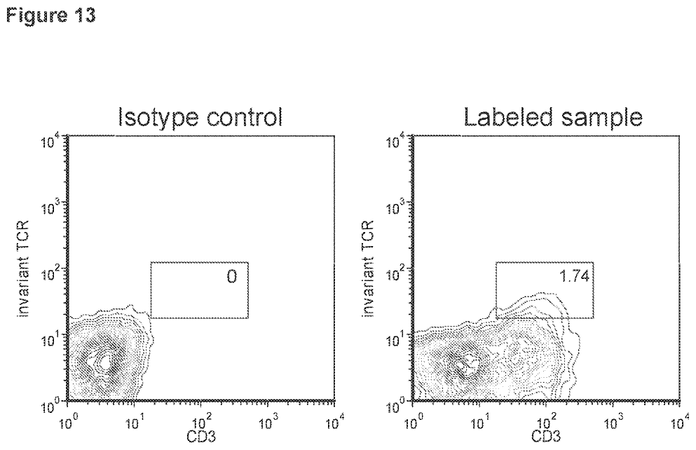

Flow cytometry. T cells were labeled with anti-human V alpha 24 J alpha 18 TCR (invariant NKT marker) conjugated to phycoerythrin (PE) (eBiosciences) to show that NKT cells are present in breast tissue from a healthy donor (FIG. 13). A FACS Calibur flow cytometer may be used to acquire the data.

Discussion

Traditional culture-based methods tend to underestimate and bias the microbial diversity in a given sample, therefore, the role of microbes in breast carcinogenesis has not been thoroughly explored. Here, next-generation sequencing techniques were used to perform a high-resolution survey of the resident breast microbiota in tumor and paired normal breast tissue from breast cancer patients. In addition, a potential association of bacterial load with levels of immune gene expression was investigated by quantifying the amount of bacteria present in healthy and tumor tissue and correlating bacterial load with the magnitude of antibacterial immune responses in the tissue.

Previous paradigms of microbes in disease point to specific pathogenic bacteria as exclusive causes. Indeed, Helicobacter pylori infection is known to promote gastric cancer and gastric mucosal-associated lymphoid tissue (MALT) lymphoma (Siman, 1997; Uemura, 2001). Reports have also linked the presence of pathogenic Escherichia coli containing pks toxicity genes with local tissue inflammation and colon carcinogenesis (Arthur, 2012). However, recent studies have revealed that the interactions between bacteria and host can be far more complex. First, microbial community composition and relative abundance of bacterial species can be contributory factors to health and disease (Turnbaugh, 2006; Turnbaugh, 2009A; Turnbaugh, 2009B). Second, not all bacteria are pathogenic; in fact, some bacteria have probiotic effects that help to maintain health status (Mazmanian, 2008). An example of this is the bacterium Bacteroidetes fragilis, a probiotic organism that protects against experimental colitis by suppressing production of the proinflammatory cytokine IL-17 in the gut (Mazmanian, 2008A; Mazmanian, 2008B). As in the gut, the presence of a specific bacterium may be beneficial in the breast as indicated above. In the study described herein, the association of S. yanoikuyae with normal breast tissue and the dramatic reduction in its abundance in corresponding tumor tissue suggests that this organism may have probiotic functions in the breast. Interestingly, S. yanoikuyae express glycosphingolipid ligands, which are potent activators of invariant NKT (iNKT) cells (Kinjo, 2005). iNKTs are important mediators of cancer immunosurveillance (Terabe, 2007) and have been reported to have an integral role in controlling breast cancer metastasis (Hix, 2011). Further studies are aimed at investigating the potential role of S. yanoikuyae in breast cancer development and progression.

In a quantitative survey of breast microbiota, the amount of bacteria was not significantly different in paired normal tissue from breast cancer patients and healthy breast tissue from healthy individuals. However, compared to both these tissues, breast tumor tissue had significantly reduced amounts of bacteria. This reduction coincided with reduced expression of one-third of antibacterial response genes surveyed. Innate immune sensors including TLR 2, 5 and 9 and antimicrobial response effectors IL-12A, BPI and MPO were all expressed at lower levels in tumors compared to healthy breast tissue. Taken together, these data suggest that bacteria may have a role in maintaining healthy breast tissue through stimulation of host inflammatory responses.

The data provided herein supports a model in which bacteria contribute to maintenance of healthy breast tissue by stimulating resident immune cells. When dysbiosis occurs, a reduction in the overall number of bacteria and/or the abundance of specific species such as S. yanoikuyae, may lead to decreased bacterial-dependent immune cell stimulation, ultimately resulting in a permissive environment for breast tumorigenesis.

The significant reduction in bacterial load found in breast tumor compared to healthy breast tissue demonstrates that bacterial load could be an additional indicator of diagnosis or staging of breast cancer. In addition, the inverse correlation between bacterial load and tumor stage implies that bacterial load might be used in conjunction with current methods to monitor the progression of breast cancer and to facilitate staging of the disease. Furthermore, the results of the studies described above may be indicate that a decrease in bacterial load in a healthy individual may be a signal of heightened breast cancer risk.

### Example 3. Breast Ducts Harbor a Microbial Community

The goal in this Example and the Examples described below was to map the microbiome of the normal and early cancerous breast duct as a basis for identifying infectious organisms which might contribute directly (affecting tumor initiation or transformation) or indirectly (by chronic inflammation) to breast carcinogenesis. By comparing the bacterial and viral diversity naturally found in the breast ducts—the tumor tissue of origin—of normal post-pubertal, premenopausal women to that of women with breast cancer limited to the duct (ductal carcinoma in situ, DCIS), the potential of an infectious etiology for the disease was explored. The information obtained from this study may have an enormous impact, transforming the current understanding of breast cancer etiology and approach to therapy, while setting the stage for a preventative therapy.

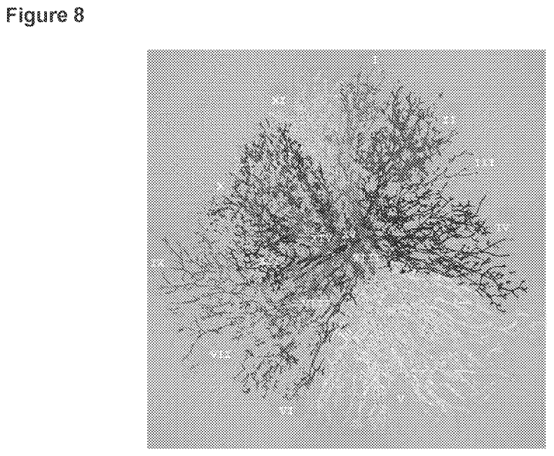

Human experimental model. One of the distinguishing factors in this Example and the Examples described below is that the research was focused on the human breast duct, in vivo. This is important because the tropism of microbes is species specific, such as HPV. In addition, the anatomy of the human breast is different than that seen in most animal models in that there are 6-8 ductal systems opening on the surface of the nipple per breast (Going, 2004; Love, 2004) (FIG. 8). The human infant spends a longer time being nourished by the breast than most other mammals and other sexual oral nipple contact is probably different among species.

Breast ductal fluid. Since all breast cancer starts in the epithelial cells lining the independent ducts, the focus in this Example and the Examples described below was on the ductal fluid as being most likely to yield relevant information on the microbiome of the breast with the least amount of contaminating human DNA. The data from this Example were obtained from nipple aspirate fluid (NAF), for its ease of collection and the fact that the two subjects tested produced NAF. However, since not all women produce NAF and its physiology is unknown, the ductal fluid may also be obtained by lavage.

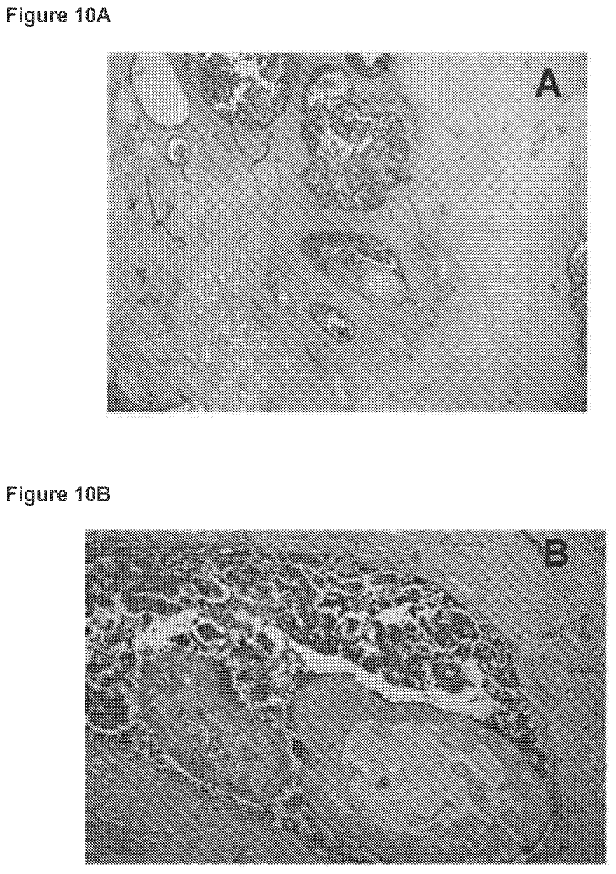

Ductal lavage (FIG. 9A) was developed by Dr. Susan Love (Dooley, 2001; Tondre, 2008) and is useful in that it can be used to interrogate the individual duct harboring ductal carcinoma in situ (DCIS). The technique for identifying the nipple orifice of the involved duct has been demonstrated in studies of intraductal therapy. Essentially, the position of the ductal orifice in the nipple correlates to the corresponding ductal system: central ducts project directly back towards the chest wall and peripheral ducts extend radially (Love, 2004). By determining whether the microcalcifications indicative of the DCIS are central or peripheral and where they are located on a clock face, the appropriate duct orifice can be identified. The procedure is monitored with ultrasound to confirm that the correct duct is cannulated. This approach has been confirmed with ductograms in subsequent neoadjuvant studies in women (Mahoney, 2009; Steams, 2011) (FIG. 9B). The ductograms and histological analysis also demonstrate that instilled fluid can traverse the entire duct, through the regions of DCIS and without extravasation even following a diagnostic core biopsy (FIGS. 10A and 10B).

Materials and Methods

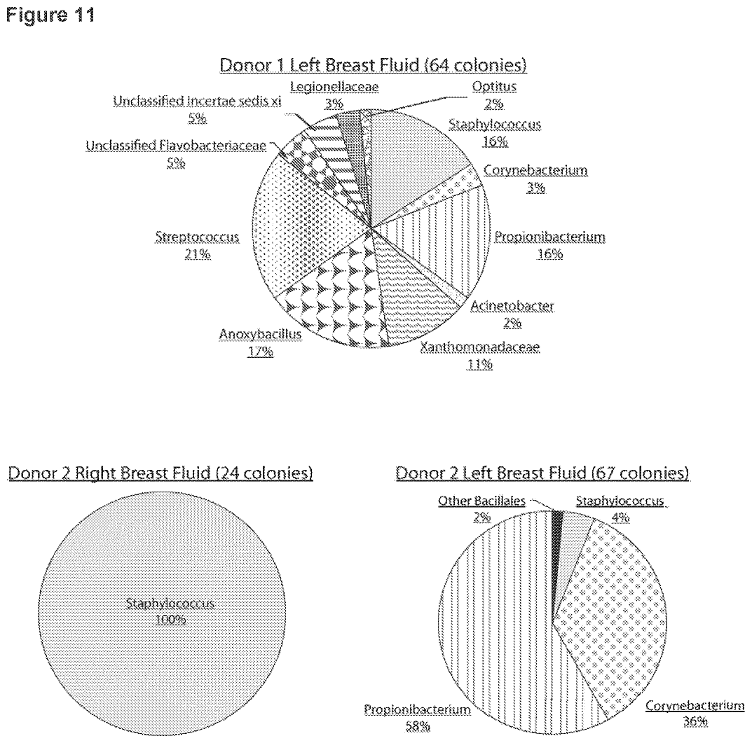

Nipple aspirate fluid collection. To determine whether microbes reside in breast ducts, the ductal fluid was probed from two subjects (Donor 1 and Donor 2) for the 16S bacterial ribosomal DNA (rDNA) gene (FIG. 11). NAF was collected using a sterile nipple aspiration technique developed by the Dr. Susan Love Research Foundation. The technique was informed by a study by the Cazzaniga group, who examined ductal fluid for 21 human papilloma virus (HPV) types in women with increased breast cancer risk. While they found a low prevalence of HPV DNA, their study demonstrated the importance of excluding cutaneous contaminants (Cazzaniga, 2008). Thus, to reduce skin contamination, the nipple and surrounding areas were sterilized with betadine prior to fluid collection. Genomic DNA was extracted from the nipple fluid as previously described (Grice, 2009). The nearly full length 16S rDNA gene was PCR-amplified, cloned and sequenced by the Sanger method. Sequences were assigned to bacterial genera based on the Ribosomal Database Project (RDP).

Extraction and amplification of bacterial DNA from saline samples stored at −80° C. Forearm and mouth swab samples in a total volume of 10 mL sterile saline were stored at 4° C. or −80° C. for 2 days. The samples were centrifuged at 3200×g for 30 minutes and genomic DNA was extracted from the pellet. Bacterial 16S rDNA primers (Forward 8F/27F; Reverse 1510R) were used to amplify the DNA by PCR.

Results

The breast duct harbors a microbial community. While the experiments described in this Example only included a small number of sequences, and thus only dominant species were detected, the data show that the bacterial diversity in the fluid from breast ducts differs from that found on the skin. In the nipple skin of Donor 1, Xanthomonadaceae was the most abundant genera found. Propionibacterium and Finegoldia were also relatively abundant, consistent with previous reports (Grice, 2009). Following application of betadine to sterilize the nipple area, residual skin flora obtained by swab was comprised of Staphylococcus (the most abundant genera found-37%), Streptophyta (18%) and Ralstonia (18%) on Donor 1.

While Donor 1 produced only a very small amount of fluid from one breast which was swabbed from the nipple, Donor 2 was able to produce nipple aspirate fluid from both breasts and several ducts. In the ductal fluid from Donor 1, Acinetobacter, Xanthomonadaceae, Staphylococcus, Streptococcus, Propionibacterium, Corynebacterium, and Flavobacteria were detected (FIG. 11), reflecting organisms also found in skin and oral microbiomes (Grice, 2009; Bik, 2010; Dewhirst, 2010; Gao, 2007; Griffen, 2011). The ductal fluid from Donor 2 has a less diverse microbiome, mainly consisting of Staphylococcus, Propionibacterium and Corenebacterium. This preliminary data indicated that the ductal fluid from normal healthy women contains a microbiome that is distinct from nipple skin, and that NAF is different between individuals and between breasts in a given person. However, this preliminary study used NAF and these findings may not be applicable to lavage of individual ducts.

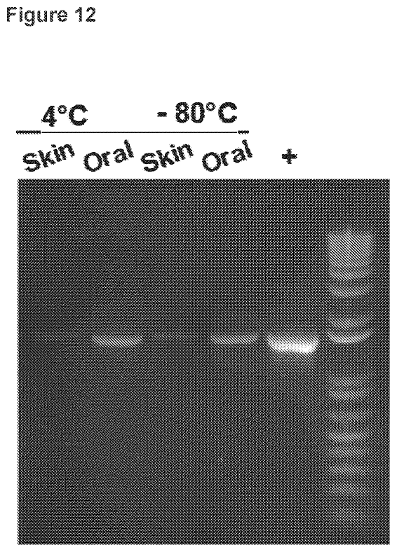

Bacterial DNA detected from saline samples stored at −80° C. detectable. The feasibility of obtaining bacterial DNA from swabbed skin or oral mucosal surfaces which were diluted in a volume similar to what would be expected from breast ductal lavage was investigated. Samples from the Serial Evaluation of Ductal Epithelium (SEDE) bank that were stored at −80° C. were also investigated to determine the ability to isolate bacterial DNA from dilute samples in saline which have been kept at −80° C. The data demonstrated that microbial DNA could be extracted from saline diluted bacteria obtained by swabbing the forearm and mouth stored at either 4° C. or −80° C. (FIG. 12).

Targeted studies for microorganisms in the breast, study of the microbiota in milk, and the data from this Example, indicate that a population of microbes resides in the ducts. Since breast cancer develops from ductal epithelium, a distinct subset of microbes residing in the ducts may exist that may contribute to breast cancer.

### Example 4. Comparison of the Bacterial Diversity of Multiple Ducts in Normal Subjects by 16S rDNA Sequencing Using the Roche/454 Platform

As described in this Example and Example 5 below, a pilot study may be conducted of multiple ducts per breast in normal women as well as multiple ducts of DCIS subjects including the duct containing DCIS to test whether breast ducts contain the same or different microbiota by the study of ductal lavage fluid. The ducts may be the same or different in normal subjects and in DCIS, but the same may not be true for both groups. This may be important in determining whether a distinct set of microbes at the site of disease is associated with DCIS in premenopausal postpubertal women. This information may be important for future studies. If ducts are the same in any given individual, future studies may be performed to sample one duct to be representative for a patient (either normal or DCIS).

Rationale and experimental design. Since the exposure of each breast to oral and skin microbes is the same, the microbiomes of the individual ducts are also likely the same, yet DCIS has been shown to be limited to one ductal system (Tot, 2005). Multiple factors likely contribute to breast carcinogenesis and it is the interaction between the microbiota and other variables unique to a given duct that may determine whether cancer develops. The data from Example 3 (FIG. 5) was generated by the study of NAF samples and suggests that the breasts within a given individual may be different, but NAF may have a different physiology than ductal lavage fluid. To establish whether the microbiomes of the ducts within and between breasts are the same or different, a pilot study of the bacterial biome may be undertaken using 16S rDNA sequencing of multiple ducts per breast by obtaining ductal lavage fluid from subsets of women in similar states of puberty and/or menopause.

Recruitment of subjects and acquisition of samples. As described in this Example, healthy premenopausal women may be recruited to undergo lavage under sterile conditions. Women with nipple piercings, previous history of breast infection or mastitis may be excluded. All subjects may also fill out a questionnaire regarding risk factors for breast cancer as well as other factors which may influence the microbial population and potential sources of microbial exposure.

### Materials and Methods

Intraductal approach for collection of breast ductal lavage fluid samples. The catheter that may be used in this procedure is described in Tondre et al (Tondre, 2008). Three ducts per breast may be sampled to determine whether the biome is uniform among ducts from a single patient. Prior to any sterilization, nipple skin may be swabbed to determine the individual's skin microbiome for comparison to the duct. Betadine may be used to sterilize the nipple skin, and the nipple may then be swabbed again to determine what potential contaminants are still present the nipple skin, and then ductal lavage may be performed. The fluid may be flash frozen in liquid nitrogen, placed in dry ice and shipped or transported to the necessary laboratory. Bactenal diversity analysis. Fluid samples may be centrifuged at 4000 g to pellet bacteria. Genomic DNA extraction may then be performed. Two variable regions of the 16S rDNA gene, V1-V3 and V3-V5, may be amplified and sequenced.

Sequencing Strategies. The 16S rDNA genes in breast ductal microbiome may be analyzed using 454/Roche sequencing platform. The current Titanium instrument generates 1 million reads per run with average read length of 400-700 bp. The samples may be prepared using degenerate PCR primers that have been developed for variable regions within the 16S rDNA gene. Two regions may be used: V1-V3 and V3-V5, to be consistent with the current protocol adapted by the Human Microbiome Project to analyze the reference sample set from ˜300 donors. Approximately 5,000 reads/sample may be obtained, which may allow for detection of the species at the abundance level as low as 0.1% with roughly five sequence reads for each variable region. Up to 96 samples may be sequenced in one run, and two runs should accommodate all 150 samples that may be analyzed. The sequences of 96 versions of each of the two region's primer pairs are available. Each of these 96 versions of a primer pair contains a sequence barcode added to the primer, and these have been vetted to ensure no bias is introduced by the addition of this short sequence. PCR may be performed on up to 96 samples each time using the 96 primer sets, the PCR products pooled, and a single library per variable region for 454 sequencing may be constructed.

Data Analysis. The resulting reads from each run may be deconvoluted into the individual samples based on the barcodes for further analysis. To classify the 16S rDNA sequences, the RDP or SILVA 16S rDNA databases may be used to determine which organisms are present in each sample. Statistical analyses, including UniFrac analysis (Caporaso, 2010) may be applied to assess whether the microbiome in different ducts are the same, whether the ducts from different breasts are the same, and whether there is a core microbiome shared by different individuals. Data from normal individuals may enable characterization of the microbiome of the breast ducts and offer insight into the diversity and variability of the microbial population among the ducts of individual women and between the ducts of different women.

Because there may be contamination issues that interfere with the collection of accurate data, measures may be instituted to prevent this, including minimizing exposure to additional microbes during sample collection and processing. For example, solutions used during collection and processing should be sterile, negative controls may be added at each step of the collection and processing, and the collection may be performed in sterile conditions, including prepping the area to be sampled. Additionally, a clean room may be used that is only used for DNA extraction purposes for this project.

### Example 5. Comparison of the Bacterial Diversity of the DCIS Containing Duct to Other Ducts in DCIS Subjects by 16S rDNA Sequencing Using the Roche/454 Platform

Rationale and experimental design. While all ducts may be the same in a given normal subject, ducts in DCIS subjects may not. Women with DCIS were chosen for the experiments described in this Example because the malignancy is an early lesion and confined to the duct which remains intact. Once breast cancer becomes invasive, the integrity of the involved ductal system is breached, and ductal lavage is no longer a reliable method for sampling the ductal fluid (Khan, 2004). Study of this small subset of patients will also allow for the development of a standardized approach to establish the most effective protocol for performing lavage on the operating table, the use of intraoperative imaging to confirm lavage of the DCIS duct, and methods for processing and shipping.

Recruitment of subjects and acquisition of samples. Ten premenopausal women with DCIS may be recruited and multiple ducts may be sampled, including the duct with DCIS. Women with nipple piercings and previous history of breast infection or mastitis may be excluded. All subjects may also fill out a questionnaire regarding risk factors for breast cancer as well as other factors which may influence the microbial population and potential sources of microbial exposure. Ten women with DCIS may undergo ductal lavage.

Materials and Methods

Intraductal approach for collection of breast ductal lavage fluid samples. Ductal lavage may be performed on women with DCIS after diagnosis but before definitive surgery. The lavage may be performed after the operative sterile field has been established in the operating room. DCIS subjects may be under anesthesia and in the sterile environment of the operating room. They may undergo lavage of the DCIS duct, confirmed with intraoperative ultrasound which can visualize the fluid, as well as at least one other duct in the same breast and one from the contralateral breast. The specimens may be processed immediately and shipped. This experiment is important to standardize the protocol of performing lavage on the operating table, integrating intraoperative imaging to confirm lavage of the DCIS duct, and processing and shipping procedures across both clinical sites in anticipation of sampling a larger set of patients such as in Example 4.

All samples in endotoxin-free physiologic saline may be coded and no protected health information will be transferred with the samples. The fluid may be flash frozen in liquid nitrogen, placed in dry ice and shipped or transported to the necessary laboratory.

Bacterial diversity analysis. Fluid samples may be centrifuged at 4000 g to pellet bacteria. Genomic DNA extraction may then be performed. Two variable regions of the 16S rDNA gene, V1-V3 and V3-V5, may be amplified and sequenced.

Sequencing Strategies. The 16S rDNA genes in breast ductal microbiome may be analyzed using 454/Roche sequencing platform. The current Titanium instrument generates 1 million reads per run with average read length of 400-700 bp. The samples may be prepared using degenerate PCR primers that have been developed for variable regions within the 16S rDNA gene. Two regions may be used: V1-V3 and V3-V5, to be consistent with the current protocol adapted by the Human Microbiome Project to analyze the reference sample set from ˜300 donors. Approximately 5,000 reads/sample may be obtained, which may allow for detection of the species at the abundance level as low as 0.1% with roughly five sequence reads for each variable region. Up to 96 samples may be sequenced in one run, and two runs should accommodate all 150 samples that may be analyzed. The sequences of 96 versions of each of the two region's primer pairs are available. Each of these 96 versions of a primer pair contains a sequence barcode added to the primer, and these have been vetted to ensure no bias is introduced by the addition of this short sequence. PCR may be performed on up to 96 samples each time using the 96 primer sets, the PCR products pooled, and a single library per variable region for 454 sequencing may be constructed.

Data Analysis. Similar to Example 4, the resulting reads from each run may be deconvoluted into the individual samples based on the barcodes for further analysis and taxonomy assignment. Statistical analyses, including UniFrac analysis (Caporaso, 2010), may be applied to assess whether the microbiome in the diseased duct is the same as in normal ducts, whether the normal ducts from DCIS patients are the same as in healthy subjects, and whether there is a core microbiome shared by diseased ducts among different DCIS patients. This analysis may enable characterization of the microbiome of the breast ducts in DCIS patients and may offer insight into the variability of the microbial population in healthy and diseased states.

Should a Surgeon have limited time under anesthesia, he or she may not be able to lavage all of the ducts proposed for DCIS subjects. In addition, the duct may be perforated (a rare complication in <10% and visible on ultrasound) and the lavage may be not just of the duct but also the stroma. This may lead to more human cells associated with the sample which could be removed by filtration (0.8 micron filter) if necessary and should not preclude valid analysis.

### Example 6. Comparison of the Bacterial Diversity in Normal Subjects and Those with DCIS by 16S Ribosomal DNA (16S rDNA) Sequencing Using Roche/454 Platform

Rationale and experimental design. The bacterial microbiome may be different in DCIS patients, and perhaps even the DCIS affected duct compared to normal subjects or normal ducts within patients with DCIS. This may be tested by performing 16S ribosomal DNA sequencing (FIG. 5) as described above in Examples 4 and 5. The data obtained from Examples 4 and 5 may help determine the exclusive criteria as well as the appropriate technique including whether one or multiple ducts should be sampled.

Recruitment of subjects and acquisition of samples. 48 premenopausal women with DCIS and 48 matched healthy women (breastfeeding, hormones and parity) may be studied. One duct per subject may be studied to identify a unique DCIS signature correcting for potential confounding factors. For DCIS subjects the DCIS-affected duct may be sampled. Women may be approached after diagnosis but before definitive surgery. All subjects may also complete a questionnaire regarding the risk factors for breast cancer as well as other factors which may influence the microbial population

48 healthy premenopasual women may also be recruited that are matched to the DCIS patients according to parity, breast feeding history and hormone use. They may undergo lavage of one duct under sterile conditions as described above in Examples 4 and 5.

Materials and Methods

Standardized lavage and collection/shipping protocols developed in Examples 4 and 5 may be used at the surgical sites. Genomic DNA may be extracted and a small amount may be used for 16S rDNA sequencing as described above in Examples 4 and 5. The remaining DNA may be used as described in Example 7 below for metagenomic sequencing.

Sequencing Strategies. Similar to that as described above in Examples 4 and 5, the 16S rDNA genes in breast ductal microbiome may be analyzed using the 454/Roche sequencing platform. Two regions, V1-V3 and V3-V5, of the 16S rDNA may be sequenced. Approximately 5,000 reads/sample may be obtained, which may detect the species at the abundance level as low as 0.1% with roughly five sequence reads for each variable region. All 96 samples may be sequenced in one run with the same strategy of multiplexing as described in Examples 4 and 5. PCR may be performed on all 96 samples using the 96 primer sets, the PCR products may be pooled, and a single library per variable region may be constructed for 454 sequencing.

Data Analysis. The resulting reads from each run may be deconvoluted for further analysis into individual samples based on the barcodes. To classify the 16S rDNA sequences, the RDP or SILVA 16S rDNA databases may be used to determine which organisms are present in each sample. Statistical analyses may be applied to assess whether certain species/phylotypes are differentially present/absent in ductal samples from normal individual and DCIS patients. Multivariate analysis may be used to compare the mean quantities of sequence reads from each operational taxonomic unit between groups to assess the roles of the main variable, normal vs. disease, in the composition of the ductal microbiome in samples. The differences in species/phylotypes between normal subjects and DCIS patients may be analyzed and compared to known bacterial strains. This analysis, comparing normal subjects with DCIS patients, may enable identification of specific organisms that are associated with the disease.

One run on the Roche/454 Life Sciences sequencer can accommodate 96 samples. Additional samples may be performed by multiplexing samples, thereby maintaining the same cost (one run can perform 96 samples, multiplex can sequence 192 samples for the same run). Multiplex may be used for up to two ducts per person; therefore, if needed, the number of subjects may be decreased if more than two ducts are queried per subject.

This study of the bacterial microbiome by 16S sequencing may provide information towards the richness (number of different species) and evenness (relative abundance of different species) in the normal versus DCIS breast duct communities.

### Example 7: Comparison of the Bacterial and Viral Metagenome from Normal Subjects and Those with DCIS by Metagenomic Sequencing

Metagenomic sequencing may provide genetic information regarding both the bacterial and viral genes present, in addition to taxonomic diversity. For example, a recent study by Turnbaugh and colleagues indicated that although in one given disease state (obesity) there was not a common group of microbes shared among all individuals, at the genomic level a clear representation of bacterial gene functions and metabolic pathways was identified (Turnbaugh, 2009A). Therefore, the data from this Example may provide information regarding the bacterial and viral microbiome of the breast duct as well as microbial genes in normal and DCIS breast ducts.

Rationale and Experimental design. The bacterial and viral microbiome may be different in DCIS patients, and perhaps even the DCIS affected duct compared to normal subjects or normal ducts within patients with DCIS. While 16S sequencing of samples collected in Example 6 may provide information on the bacterial diversity of the normal and DCIS subjects (FIG. 5), metagenomic sequencing may provide even more comprehensive data including both bacterial and viral diversity information. Therefore, over half of each sample collected from Example 6 may be utilized to perform metagenomic sequencing.

Recruitment of subjects and acquisition of samples. Samples collected in Example 6 may be studied as described above.

Materials and Methods

Metagenomic sequencing to identify bacterial and viral diversity. DNA extracted for experiments as described in Example 6 above may be used for the metagenomic sequencing in the present Example.

Sequencing Strategies. 100-600 species level operational taxonomic units have been found in the human milk (Hunt, 2011). Among them, 12 genera were shared by all the samples studied. In the study performed in Example 3, 1 to 11 genera were found in different samples (see FIG. 5). On the basis of these data, it was estimated that approximately 100-200 microbial species may be found in breast ducts. This translates to a microbiome size of 300 Mb-600 Mb. Each sample may be sequenced using Solexa/lllumina high-throughput sequencing technology. Illumina HiSeq platform routinely generates 100 million reads per lane, 100 billion bp per run, with 100 bp-long reads. The ultra high-throughput of the sequencing technology increases the accuracy of the reads and metagenome coverage, helps the partial assembly of abundant genomes, increases the confidence in gene identification, as well as enables the quantification of the enrichment of functional genes in samples. Based on the experience working with stool samples, which require about 10 billion bp of sequence to achieve at least 2× coverage of the minor species (1% abundance), the sequencing depth required for the ductal samples was estimated. Each sample may be sequenced in one HiSeq lane. This may give 15-30× coverage of the microbiome.

Bioinformac Analysis: There are several steps in the sequence data analysis which are outlined below.

1. The metagenome sequence reads from each sample may be assembled first. It is expected to be able to partially assemble the genomes of the abundant species into large contigs.

2. The contigs and sequence fragments may be compared to multiple sequence databases, including Human Microbiome Project (HMP) reference strain database, non-redundant database (nr), metagenomic databases (CAMERA, IMG, etc.) to annotate the functions of the coding sequences. In particular, the HMP database is relevant to this Example and may be used.

3. The genetic differences between samples may be identified: normal versus DCIS. This includes two aspects: gene composition and abundance. The common genes or common variations in gene abundance between the groups may be determined as the metagenomic signatures for each state.

Gene composition. Existing methods are being improved and new computational methods are being developed to compare metagenome samples, which are not fully assembled in most cases.

Gene abundance. An approach similar to RNA-seq data analysis (Wilhelm, 2009) may be used, but instead of analyzing transcript abundance in one genome, the gene abundance may be analyzed in metagenomes. The copy number of each gene or genetic element may be computed from the sequencing reads and normalized by reads per Kb per million reads (RPKM) (Dean, 2001).

Multiple ways of defining the “same gene”. In this Example, two genes may be defined as the same by the following criteria: 1) they have a sequence similarity >50% in the overlapping region: 2) the minimum overlapping region is 100 bp; 3) they have the same function annotation based on BLAST result. This definition cannot exclude the possibility that two genes from different organisms may be identified as the same gene, such as in the case of well-conserved genes or horizontally transferred genes. However, this would not significantly affect the identification of functional signatures of the metagenome, because certain gene functions, rather than species origin, may play an important role in the pathogenesis. The recent study of the human gut microbiome also provides support that certain functional groups of genes rather than microbial species are shared among diseased state (Turnbaugh, 2009A).

In an alternative embodiment, bacterial components may be filtered by filtering the fluid with a 0.45 micron filter. The viral particles may also be concentrated by ultracentrifugation (50,000 g×3 hours at 10° C.) or cesium chloride gradient. The sequencing data generated from the Illumina sequencer require computational capacity and capability. Further, once a matured protocol and analysis pipeline of the microbiome in the breast duct is established, RNA-seq may be performed to examine the expressed functions of the microbiome as well as RNA viruses.

By including human cells from the ductal lavage fluid, lysis and bead beating should be able to release the genomic content of intracellular viruses.

With respect to the amount of genomic DNA needed for Illumina library construction, the current protocol has been routinely used to construct libraries for Illumina sequencing runs with 100 ng genomic DNA, and have used as low as 10 ng. From the study described in Example 3 of the NAF samples, on average 10 ng genomic DNA per sample was obtained. In the present Example, the lavage samples may contain a similar amount of microbes as the NAF samples; thus, the amount of DNA extracted should be adequate for sequencing. Alternatively, whole genome amplification using the multiple displacement amplification (MDA) approach may also be utilized. MDA uses 029 DNA polymerase to amplify whole genomes (GenomiPhi DNA amplification kit by Amersham Biosciences) (Dean, 2001; Detter, 2002). This polymerase has also been used for whole-genome amplification of bacterial isolates (Detter, 2002; Raghunathan, 2005) and in studies of metagenomic samples (Abultencia, 2006). Because the method is extremely sensitive, it is important to perform the experiments in exceptionally clean conditions and with negative controls. To minimize artifacts, whole genome amplification may be performed on samples from both normal individuals and DCIS patients.

In addition, previous data show that the genomic DNA extracted from skin samples contains less than 10% of human DNA. The high coverage of the Illumina sequencing reads should overcome this issue without reducing the number of microbial DNA reads significantly. The human DNA reads may be filtered out later computationally according to the standard HMP protocol. In the event that the human DNA contamination may be an issue, human cells may be separated by modifying established protocols using filtration (0.8 micron and 0.45 micron filters in series), then purified and concentrated using a cesium chloride (CsCl) gradient to remove free DNA and any remaining cellular material (Willner, 2011; Willner, 2009). The presence of virus-like particles (VLPs) and the absence of microbial contamination may be verified by epifluorescence microscopy using SYBR® Gold (Thurber, 2009).

The results from these experiments may identify the microbes residing in the breast ducts of healthy individuals and provide a comparison to those found in DCIS patients. This may allow for a determination upon whether there is a disease-associated signature of the microbiome in affected ducts with early breast cancer.

### Example 8. Determination of Microbiome Signatures from High Risk Women Whose Subsequent Outcome of Developing Breast Cancer is Known

Rationale and experimental design. The value of next generation sequencing for the identification of microorganisms and their gene products provides a wealth of information and allows for a comprehensive investigation of the microbiome in the ducts. However, given the volume of data and cost of technology, this technique is not practical for large population studies required to establish association with disease and causality.

There may be a distinct bacterial and/or viral microbiome associated with breast cancer and these microbes may be present in ductal fluid prior to the development or detection of breast cancer. Thus, to test whether the distinct DCIS microbiome identified in Example 7 is present prior to breast cancer diagnosis in high risk subjects, the distinct microbiome signature identified in the previous Examples that are associated with DCIS in banked fluid may be compared from high risk women who did and did not develop breast cancer. DNA for use in this determination may be isolated from ductal lavage fluid or nipple aspirate fluid.

Statistical Analysis. The analysis for the qPCR data will seek to determine whether these metagenomic signatures can be used as classifiers to differentiate DCIS samples from normal samples. Fisher's Exact test or chi-square test may be used to compare the frequencies of each allele of each sequence between the groups. Since combinations of metagenomic signatures may be better predictors, logistic regression models may be used to identify combinations that best predict sample identity.

### REFERENCES

The references, patents and published patent applications listed below, and all references cited in the specification above are hereby incorporated by reference in their entirety, as if fully set forth herein.
Abulencia C B, Wyborski D L, Garcia J A, Podar M, Chen W, Chang S H, et al. Environmental whole-genome amplification to access microbial populations in contaminated sediments. Appl Environ Microbiol. 2006; 72(5):3291-301. PMCID: 1472342.
Akil N, Yasmeen A, Kassab A, Ghabreau L, Darnel A D, Al Moustafa A E. High-risk human papillomavirus infections in breast cancer in Syrian women and their association with Id-1 expression: a tissue microarray study. Br J Cancer. 2008; 99(3):404-7.
Arthur J C, Perez-Chanona E, Muhlbauer M, Tomkovich S, Uronis J M, et al. (2012) Intestinal Inflammation Targets Cancer-Inducing Activity of the Microbiota. Science 338: 120-123.
Bartsich S, Ascherman J A, Whittier S, Yao C A, Rohde C. The breast: a clean-contaminated surgical site. Aesthet Surg J. 2011; 31(7):802-6.
Bebok Z, Markus B, Nemeth P. Prognostic relevance of transforming growth factor alpha (TGF-alpha) and tumor necrosis factor alpha (TNF-alpha) detected in breast cancer tissues by immunohistochemistry. Breast Cancer Res Treat. 1994; 29(3):229-35.
Bhandare D. Endocrine Biomarkers in Ductal Lavage Samples from Women at High Risk for Breast Cancer. Cancer Epidemiology Biomarkers & Prevention. 2005:14(11):2620-7.
Bik E M, Long C D, Armitage G C, Loomer P, Emerson J, Mongodin E F, et al. Bacterial diversity in the oral cavity of 10 healthy individuals. ISME J. 4(8):962-74. PMCID: 2941673.
Bonnet M, Guinebretiere J M, Kremmer E, Grunewald V, Benhamou E, Contesso G, et al. Detection of Epstein-Barr virus in invasive breast cancers. J Natl Cancer Inst. 1999; 91(16):1376-81.
Cabrera-Rubio R, Collado M C, Laitinen K, Salminen S, Isolauri E, et al. (2012) The human milk microbiome changes over lactation and is shaped by maternal weight and mode of delivery. The American Journal of Clinical Nutrition 96: 544-551.
Caporaso J G, Kuczynski J, Stombaugh J, Bittinger K, Bushman F D, Costello E K, et al. QIIME allows analysis of high-throughput community sequencing data. Nat Methods. 7(5):335-6. PMCID: 3156573.
Casano A, Tondre J, Mills D, Love S M. Atypia on ductal lavage: A reproducible marker of risk? 2008 Frontiers in Cancer Prevention Research; National Harbor, Md. 2008.
Castellarin M, Warren R, Freeman J D, Dreolini L, Krzywinski M, et al. (2012) Fusobacterium nucleatum infection is prevalent in human colorectal carcinoma. Genome Research 22: 299-306.
Castillo M, Martin-Orue S M, Manzanilla E G, Badiola I, Martin M, et al. (2006) Quantification of total bacteria, enterobacteria and lactobacilli populations in pig digesta by real-time PCR. Veterinary Microbiology 114: 165-170.
Caygill C P, Braddick M, Hill M J, Knowles R L, Sharp J C. The association between typhoid carriage, typhoid infection and subsequent cancer at a number of sites. EurJCancer Prev. 1995; 4(2):187-93.
Cazzaniga M, Gheit T, Casadio C, Khan N, Macis D, Valenti F, et al. Analysis of the presence of cutaneous and mucosal papillomavirus types in ductal lavage fluid, milk and colostrum to evaluate its role in breast carcinogenesis. Breast Cancer Research and Treatment. 2008; 114(3).
Chatterton R T, Jr., Geiger A S, Khan S A, Helenowski I B, Jovanovic B D, Gann P H. Variation in estradiol, estradiol precursors, and estrogen-related products in nipple aspirate fluid from normal premenopausal women. Cancer Epidemiol Biomarkers Prev. 2004; 13(6):928-35.
Chatterton R T, Parker N P, Habe-Evans M, Bryk M, Scholtens D M, Khan S A. Breast Ductal Lavage for Assessment of Breast Cancer Biomarkers. Hormones and Cancer. 2010; 1(4):197-204.
Clavel T, Henderson G, Alpert C A, Philippe C, Rigottier-Gois L, Dore J, et al. Intestinal bacterial communities that produce active estrogen-like compounds enterodiol and enterolactone in humans. Appl Environ Microbiol. 2005; 71(10):6077-85.
Colditz G A, Hankinson S E, Hunter D J, Willett W C, Manson J E, Stampfer M J, et al. The use of estrogens and progestins and the risk of breast cancer in postmenopausal women. N Engl JMed. 1995; 332(24):1589-93.
Dean F B, Nelson J R, Giesler T L, Lasken R S. Rapid amplification of plasmid and phage DNA using Phi 29 DNA polymerase and multiply-primed rolling circle amplification. Genome Res. 2001; 11(6):1095-9. PMCID: 311129.
DeSantis, T. Z. et al. Greengenes, a chimera-checked 16S rRNA gene database and workbench compatible with ARB. Appl Environ Microbiol 72, 5069-5072 (2006).
Detter J C, Jett J M, Lucas S M, Dalin E, Arellano A R, Wang M, et al. Isothermal strand-displacement amplification applications for high-throughput genomics. Genomics. 2002:80(6):691-8.
Dewhirst F E, Chen T, Izard J, Paster B J, Tanner A C, Yu W H, et al. The human oral microbiome. J Bacteriol. 192(19):5002-17. PMCID: 2944498.
Dong B, Kim S, Hong S, Das Gupta J, Malathi K, Klein E A, et al. An infectious retrovirus susceptible to an IFN antiviral pathway from human prostate tumors. Proc Natl Acad Sci USA. 2007; 104(5):1655-60. PMCID: 1776164.
Dooley W C, Ljung B M, Veronesi U, Cazzaniga M, Elledge R M, O'Shaughnessy J A, et al. Ductal lavage for detection of cellular atypia in women at high risk for breast cancer. J Natl Cancer Inst. 2001; 93(21):1624-32.
Durst M, Gissmann L, Ikenberg H, zur Hausen H. A papillomavirus DNA from a cervical carcinoma and its prevalence in cancer biopsy samples from different geographic regions. Proc Natl Acad Sci USA. 1983; 80(12):3812-5. PMCID: 394142.
Edgar, R. C. Search and clustering orders of magnitude faster than BLAST. Bioinformatics 26, 2460-2461.
Etkind P, Du J, Khan A, Pillitteri J, Wiernik P H. Mouse mammary tumor virus-like ENV gene sequences in human breast tumors and in a lymphoma of a breast cancer patient. Clin Cancer Res. 2000; 6(4):1273-8.
Ferreri A J, Guidoboni M, Ponzoni M, De C C, Dell'Oro S, Fleischhauer K, et al. Evidence for an association between Chlamydia psittaci and ocular adnexal lymphomas. J Natl Cancer Inst. 2004; 96(8):586-94.
Fina F, Romain S, Ouafik L, Palmari J, Ben A F, Benharkat S, et al. Frequency and genome load of Epstein-Barr virus in 509 breast cancers from different geographical areas. Br J Cancer. 2001; 84(6):783-90.
Ford C E, Tran D, Deng Y, Ta V T, Rawlinson W D, Lawson J S. Mouse mammary tumor virus-like gene sequences in breast tumors of Australian and Vietnamese women. Clin Cancer Res. 2003; 9(3):1118-20.
Gao Z, Tseng C H, Pei Z, Blaser M J. Molecular analysis of human forearm superficial skin bacterial biota. Proc Natl Acad Sci USA. 2007; 104(8):2927-32. PMCID: 1815283.
Glaser S L, Ambinder R F, DiGiuseppe J A, Horn-Ross P L, Hsu J L. Absence of Epstein-Barr virus EBER-1 transcripts in an epidemiologically diverse group of breast cancers. Int J Cancer. 1998; 75(4):555-8.
Going J J, Moffat D F. Escaping from Flatland: clinical and biological aspects of human mammary duct anatomy in three dimensions. The Journal of Pathology. 2004; 203(1):538-44.
Gopalkrishna V, Singh U R, Sodhani P, Sharma J K, Hedau S T, Mandal A K, et al.
Absence of human papillomavirus DNA in breast cancer as revealed by polymerase chain reaction. Breast Cancer Res Treat. 1996; 39(2):197-202.
Grice E A, Kong H H, Conlan S, Deming C B, Davis J, Young A C, et al. Topographical and temporal diversity of the human skin microbiome. Science. 2009; 324(5931):1190-2. PMCID: 2805064.
Griffen A L, Beall C J, Firestone N D, Gross E L, Difranco J M, Hardman J H, et al. CORE: a phylogenetically-curated 16S rDNA database of the core oral microbiome. PLoS One. 6(4):e19051. PMCID: 3081323.
Heng B, Glenn W K, Ye Y, Tran B, Delprado W, Lutze-Mann L, et al. Human papilloma virus is associated with breast cancer. Br J Cancer. 2009; 101(8):1345-50.
Hix L M, Shi Y H, Brutkiewicz R R, Stein P L, Wang C-R, et al. (2011) CD1d-Expressing Breast Cancer Cells Modulate NKT Cell-Mediated Antitumor Immunity in a Murine Model of Breast Cancer Metastasis. PLoS ONE 6: e20702.
Holmes M D, Chen W Y, Li L, Hertzmark E, Spiegelman D, Hankinson S E. Aspirin intake and survival after breast cancer. J Clin Oncol. 28(9):1467-72.
Hunt K M, Foster J A, Fomey L J, Schutte U M, Beck D L, Abdo Z, et al. Characterization of the diversity and temporal stability of bacterial communities in human milk. PLoS One. 6(6):e21313. PMCID: 3117882.
Isaacson P G, Du M Q. MALT lymphoma: from morphology to molecules. Nat Rev Cancer. 2004; 4(8):644-53.
Jemal A, Siegel R, Xu J, Ward E (2010) Cancer statistics. C A Cancer J Clin 60: 277-300.
Juttner S, Cramer T, Wessler S, Walduck A, Gao F, Schmitz F, et al. Helicobacter pylori stimulates host cyclooxygenase-2 gene transcription: critical importance of MEK/ERK-dependent activation of USF1/-2 and CREB transcription factors. Cell Microbiol. 2003; 5(11):821-34.
Khan S A, Wiley E L, Rodriguez N, Baird C, Ramakrishnan R, Nayar R, et al. Ductal lavage findings in women with known breast cancer undergoing mastectomy. J Natl Cancer Inst. 2004; 96(20):1510-7.
King B L, Love S M, Rochman S, Kim J A. The Fourth International Symposium on the Intraductal Approach to Breast Cancer, Santa Barbara, Calif., 10-13 Mar. 2005. Breast Cancer Research. 2005; 7(5):198.
Kinjo Y, Wu D, Kim G, Xing G-W, Poles M A, et al. (2005) Recognition of bacterial glycosphingolipids by natural killer T cells. Nature 434: 520-525.
Klitgaard, K., Molbak, L., Jensen, T. K., Lindboe, C. F. & Boye, M. Laser capture microdissection of bacterial cells targeted by fluorescence in situ hybridization. Biotechniques 39, 864-868 (2005).
Kostic A D, Gevers D, Pedamallu C S, Michaud M, Duke F, et al. (2011) Genomic analysis identifies association of Fusobacterium with colorectal carcinoma. Genome Research 22: 292-298.
Koutsky L A, Ault K A, Wheeler C M, Brown D R, Barr E, Alvarez F B, et al. A controlled trial of a human papillomavirus type 16 vaccine. N Engl J Med. 2002; 347(21):1645-51.
Kroupis C, Markou A, Vourlidis N, Dionyssiou-Asteriou A, Lianidou E S. Presence of high-risk human papillomavirus sequences in breast cancer tissues and association with histopathological characteristics. Clin Biochem. 2006; 39(7):727-31.
Lespagnard L, Cochaux P, Larsimont D, Degeyter M, Velu T, Heimann R. Absence of Epstein-Barr virus in medullary carcinoma of the breast as demonstrated by immunophenotyping, in situ hybridization and polymerase chain reaction. Am J Clin Pathol. 1995; 103(4):449-52.
Ley R E, Turnbaugh P J, Klein S, Gordon J I (2006) Microbial ecology: Human gut microbes associated with obesity. Nature 444: 1022-1023.
Lin S-Y, Shen F-T, Lai W-A, Zhu Z-L, Chen W-M, et al. (2011) Sphingomonas formosensis sp. nov., a polycyclic aromatic hydrocarbon-degrading bacterium isolated from agricultural soil. International Journal of Systematic and Evolutionary Microbiology 62: 1581-1586.
Lindel K, Forster A, Altermatt H J, Greiner R, Gruber G. Breast cancer and human papillomavirus (HPV) infection: no evidence of a viral etiology in a group of Swiss women. Breast. 2007; 16(2):172-7.
Love S M, Barsky S H. Anatomy of the nipple and breast ducts revisited. Cancer. 2004; 101(9):1947-57.
Love S M, Ruffell B, Nguyen T, Mills D, Coussens L M. Gene Expression of Immune Mediators within Nipple Aspirate Fluid and Ductal Lavage from Normal and Cancerous Breasts. San Antonio Breast Cancer Symposium; Dec. 6, 2011; San Antonio, Tex. 2011.
Luperchio S A, Schauer D B. Molecular pathogenesis of Citrobacter rodentium and transmissible murine colonic hyperplasia. Microbes infect. 2001; 3(4):333-40.
Luqmani Y, Shousha S. Presence of epstein-barr-virus in breast-carcinoma. Int J Oncol. 1995:6(4):899-903.
Madigan M P, Ziegler R G, Benichou J, Byrne C, Hoover R N (1995) Proportion of breast cancer cases in the United States explained by well-established risk factors. J Natl Cancer Inst 87: 1681-1685.
Mahoney M, Mills D, Love S. Intraductal therapy of DCIS with liposomal doxorubicin: a preoperative trial in rural California. BMC Proceedings. 2009; 3(Suppl 5):S31.
Mazmanian S K (2008) Capsular polysaccharides of symbiotic bacteria modulate immune responses during experimental colitis. J Pediatric Gastroenterol Nutr 46: E12-12 (Manzmanian, 2008A).
Mazmanian S K, Round J L, Kasper D L (2008) A microbial symbiosis factor prevents intestinal inflammatory disease. Nature 453: 620-625 (Manzmanian, 2008B).
McCall S A, Lichy J H, Bijwaard K E, Aguilera N S, Chu W S, Taubenberger J K. Epstein-Barr virus detection in ductal carcinoma of the breast. J Natl Cancer Inst. 2001; 93(2):148-50.
Mills D, Gordon E J, Casano A, Lahti S M, Nguyen T, Preston A, et al. The physiology of the normal human breast: an exploratory study. J Physiol Biochem. 2011; 67(4):621-7.
Monney L, Sabatos C A, Gaglia J L, Ryu A, Waldner H, Chemova T, et al. Th1-specific cell surface protein Tim-3 regulates macrophage activation and severity of an autoimmune disease. Nature. 2002; 415(6871):536-41.
Mortazavi A, Williams B A, McCue K, Schaeffer L, Wold B. Mapping and quantifying mammalian transcriptomes by RNA-Seq. Nat Methods. 2008; 5(7):621-8.
Munoz N, Bosch F X, de Sanjose S, Tafur L, Izarzugaza I, Gili M, et al. The causal link between human papillomavirus and invasive cervical cancer: a population-based case-control study in Colombia and Spain. Int J Cancer. 1992; 52(5):743-9.
Naik S, Bouladoux N, Wilhelm C, Molloy M J, Salcedo R, et al. (2012) Compartmentalized Control of Skin Immunity by Resident Commensals. Science 337:1115-1119.
Naito Y, Yoshikawa T. Molecular and cellular mechanisms involved in Helicobacter pylori-induced inflammation and oxidative stress. Free Radic Biol Med. 2002; 33(3):323-36.
Newman J V, Kosaka T, Sheppard B J, Fox J G, Schauer D B. Bacterial infection promotes colon tumorigenesis in Apc(Min/+) mice. J Infect Dis. 2001; 184(2):227-30.
Nishio T, Yoshikura T, Itoh H (1997) Detection of Methylobacterium species by 16S rRNA gene-targeted PCR. Applied and Environmental Microbiology 63: 1594-1597.
Petrakis N L. Physiologic, biochemical, and cytologic aspects of nipple aspirate fluid. Breast Cancer Res Treat. 1986; 8(1):7-19.
Pittet B, Montandon D, Pittet D (2005) Infection in breast implants. The Lancet Infectious Diseases 5: 94-106.
Raghunathan A, Ferguson H R, Jr., Bomarth C J, Song W, Driscoll M, Lasken R S. Genomic DNA amplification from a single bacterium. Appl Environ Microbiol. 2005; 71(6):3342-7. PMCID: 1151817.
Ransjo U, Asplund O A, Gylbert L, Jurell G. Bacteria in the female breast. Scand J Plast Reconstr Surg. 1985; 19(1):87-9.
Rao V P, Poutahidis T, Ge Z, Nambiar P R, Boussahmain C, Wang Y Y, et al. Innate immune inflammatory response against enteric bacteria Helicobacter hepaticus induces mammary adenocarcinoma in mice. Cancer Res. 2006; 66(15):7395-400.
Rentz, J. A., Alvarez, P., Schnoor, J. L., Benzo[a]pyrene degradation by Sphonigomonas yanoikuyae JAR02. Environmental Pollution, 151: 669-677 (2008).
Rochman S, Mills D, Kim J, Kuerer H, Love S. State of the Science and the Intraductal Approach for Breast Cancer: Proceedings Summary of The Sixth International Symposium on the Intraductal Approach To Breast Cancer Santa Monica, Calif., 19-21 Feb. 2009. BMC Proceedings. 2009; 3(Suppl 5):11.
Rossini A, Rumio C, Sfondrini L, Tagliabue E, Morelli D, et al. (2006) Influence of Antibiotic Treatment on Breast Carcinoma Development in Proto-neu Transgenic Mice. Cancer Research 66: 6219-6224.
Schloss, P. D. et al. Introducing mothur: open-source, platform-independent, community-supported software for describing and comparing microbial communities. Appl Environ Microbiol 75, 7537-7541 (2009).
Schwarz E, Freese U K, Gissmann L, Mayer W, Roggenbuck B, Stremlau A, et al. Structure and transcription of human papillomavirus sequences in cervical carcinoma cells. Nature. 1985; 314(6006):111-4.
Seo, J., Keum, Y., Li, Q. Bacterial Degradation of Aromatic Compounds. Int. J. Environ. Res. Public Health, 6, 278-309 (2009).
Siman J H, Forsgren A, Berglund G, Floren C H. Association between Helicobacter pylori and gastric carcinoma in the city of Malmo, Sweden. A prospective study. Scand J Gastroenterol. 1997; 32(12):1215-21.
Steams V, Mori T, Jacobs L K, Khouri N F, Gabrielson E, Yoshida T, et al. Preclinical and clinical evaluation of intraductally administered agents in early breast cancer. Sci Transl Med. 2011; 3(106):106ra8.
Steele V E, Moon R C, Lubet R A, Grubbs C J, Reddy B S, Wargovich M, et al. Preclinical efficacy evaluation of potential chemopreventive agents in animal carcinogenesis models: methods and results from the NCI Chemoprevention Drug Development Program. J Cell Biochem Suppl. 1994; 20:32-54.
Suzich J A, Ghim S J, Palmer-Hill F J, White W I, Tamura J K, Bell J A, et al. Systemic immunization with papillomavirus L1 protein completely prevents the development of viral mucosal papillomas. Proc Natl Acad Sci USA. 1995; 92(25):11553-7. PMCID: 40440.
Terabe M, Berzofsky J A (2007) NKT cells in immunoregulation of tumor immunity: a new immunoregulatory axis. Trends Immunol 28: 491-496.
Thornton J W, Argenta L C, McClatchey K D, Marks M W. Studies on the endogenous flora of the human breast. Ann Plast Surg. 1988; 20(1):39-42.
Thurber R V, Haynes M, Breitbart M, Wegley L, Rohwer F. Laboratory procedures to generate viral metagenomes. Nat Protoc. 2009; 4(4):470-83.
Tondre J, Nejad M, Casano A, Mills D, Love S. Technical enhancements to breast ductal lavage. Ann Surg Oncol. 2008:15(10):2734-8.
Tot T. DCIS, cytokeratins, and the theory of the sick lobe. Virchows Archiv. 2005; 447(1):1-8.
Turnbaugh P J, Ley R E, Mahowald M A, Magrini V, Mardis E R, et al. (2006) An obesity-associated gut microbiome with increased capacity for energy harvest. Nature 444:1027-1131.
Turnbaugh P J, Hamady M, Yatsunenko T, Cantarel B L, Duncan A, Ley R E, et al. A core gut microbiome in obese and lean twins. Nature. 2009; 457(7228):480-4. PMCID: 2677729 (Turnbaugh, 2009A).
Turnbaugh P J, Ridaura V K, Faith J J, Rey F E, Knight R, et al. (2009) The Effect of Diet on the Human Gut Microbiome: A Metagenomic Analysis in Humanized Gnotobiotic Mice. Science Translational Medicine 1: 6ra14 (Turnbaugh, 2009B).
Twelves D, Nerurkar A, Osin P, Ward A, Isacke C M, Gui G P H. The anatomy of fluid-yielding ducts in breast cancer. Breast Cancer Research and Treatment. 2011.
Uemura N, Okamoto S, Yamamoto S, Matsumura N, Yamaguchi S, Yamakido M, et al. Helicobacter pylori infection and the development of gastric cancer. N Eng J Med. 2001; 345(11):784-9.
Urisman A, Molinaro R J, Fischer N, Plummer S J, Casey G, Klein E A, et al. Identification of a novel Gammaretrovirus in prostate tumors of patients homozygous for R462Q RNASEL variant. PLoS Pathog. 2006; 2(3):e25. PMCID: 1434790
Velicer C M, Heckbert S R, Lampe J W, Potter J D, Robertson C A, et al. (2004) Antibiotic use in relation to the risk of breast cancer. JAMA 291: 827-835.
Wang Y, Go V, Holland J F, Melana S M, Pogo B G. Expression of mouse mammary tumor virus-like env gene sequences in human breast cancer. Clin Cancer Res. 1998; 4(10):2565-8.
Wang Y, Holland J F, Bleiweiss I J, Melana S, Liu X, Pelisson I, et al. Detection of mammary tumor virus env gene-like sequences in human breast cancer. Cancer Res. 1995; 55(22):5173-9.
Wilhelm B T, Landry J R. RNA-Seq-quantitative measurement of expression through massively parallel RNA-sequencing. Methods. 2009; 48(3):249-57.
Willner D, Furlan M, Haynes M, Schmieder R, Angly F E, Silva J, et al. Metagenomic analysis of respiratory tract DNA viral communities in cystic fibrosis and non-cystic fibrosis individuals. PLoS One. 2009; 4(10):e7370. PMCID: 2756586.
Willner D, Furlan M, Schmieder R, Grasis J A, Pride D T, Relman D A, et al. Metagenomic detection of phage-encoded platelet-binding factors in the human oral cavity. Proc Natl Acad Sci USA. 108 Suppl 1:4547-53. PMCID: 3063595.
zur Hausen H. Novel human polyomaviruses-re-emergence of a well known virus family as possible human carcinogens. Int J Cancer. 2008; 123(2):247-50.

What is claimed is:

1. A method of treating breast cancer comprising:
administering a therapeutically effective dose of a probiotic organism or its functional components to a subject suffering from breast cancer via ductal lavage or injection; wherein the probiotic organism or its functional components comprises Sphingomonas yanoikuyae.

2. The method of claim 1, further comprising administering a therapeutic agent in combination with the probiotic organism.

3. A method of treating breast cancer comprising: administering via ductal lavage or injection a therapeutically effective dose of a probiotic organism to a subject exposed to an environmental source of polycyclic aromatic hydrocarbons (PAHs), wherein the treatment is a prophylactic treatment to delay development of breast cancer; wherein the probiotic organism comprises Sphingomonas yanoikuyae.

4. The method of claim 3, wherein the environmental source of PAHs is selected from coal burners, fuel or cigarette smoke.

5. A method of treating breast cancer comprising:
determining an amount of Sphingomonas DNA in a test sample from a subject suffering from breast cancer; and
administering Sphingomonas yanoikuyae to the subject via ductal lavage or injection when the amount of Sphingomonas DNA in a test sample is significantly decreased compared to a control sample.

6. The method of claim 5, wherein the test sample is a tumor tissue sample or a ductal fluid sample.

7. The method of claim 5, wherein the amount of bacterial DNA is determined by an amplification technique, a quantification technique, a sequencing technique, or a hybridization technique.

8. The method of claim 7, wherein the amount of bacterial DNA is determined by quantitative PCR, real time PCR, digital PCR, in-situ hybridization, cDNA microarrays, immunohistochemistry, immunofluorescence, or massively parallel sequencing.

9. The method of claim 5, wherein the Sphingomonas yanoikuyae degrades an organic molecule having at least one carbon ring.


SEQUENCE LISTING (6 sequences)

SEQ ID NO: 1 - DNA, 22 bp, Artificial Sequence
  Synthetic 63F forward primer
gcaggcctaacacatgcaagtc

SEQ ID NO: 2 - DNA, 20 bp, Artificial Sequence
  Synthetic 355R reverse primer
ctgctgcctcccgtaggagt

SEQ ID NO: 3 - DNA, 18 bp, Artificial Sequence
  Synthetic Sph-spt 694F forward primer
gagatcgtccgcttccgc

SEQ ID NO: 4 - DNA, 18 bp, Artificial Sequence
  Synthetic Sph-spt 983R reverse primer
ccgaccgatttggagaag

SEQ ID NO: 5 - DNA, 19 bp, Artificial Sequence
  Synthetic 5F forward primer
cttgagtatggtagaggtt

SEQ ID NO: 6 - DNA, 19 bp, Artificial Sequence
  Synthetic 8R reverse primer
caaatctctctgggtaaca